(19) United States
(12) Patent Application Publication
(10) Pub. No.: US 2025/0308669 A1
(43) Pub. Date: Oct. 2, 2025

(54) ABSOLUTE PERFUSION RESERVE

(71) Applicant: Washington University, St. Louis, MO (US)

(72) Inventor: Scott BUGENHAGEN, St. Louis, MO (US)

(73) Assignee: Washington University, St. Louis, MO (US)

(21) Appl. No.: 18/862,936

(22) PCT Filed: May 2, 2023
(86) PCT No.: PCT/US2023/066510

§ 371 Date: Nov. 4, 2024

Related U.S. Application Data
(60) Provisional application No. 63/337,607, filed on May 2, 2022.

(51) Int. Cl. G16H 20/40; G06N 3/0464; G16B 5/00; G16H 50/50

(52) U.S. Cl. CPC G16H 20/40; G06N 3/0464; G16B 5/00; G16H 50/50

(57) ABSTRACT

Methods and systems for the diagnosis and selection for treatment of a coronary artery stenosis based on a multi-level perfusion model of the cardiovascular system are disclosed. The method further includes estimating an occluded myocardial perfusion using the multi-level perfusion model, modifying the 3D epicardial mesh to represent the treatment of the stenosis, estimating a post-treatment myocardial perfusion using the modified multi-level cardiac perfusion model, estimating an absolute perfusion reserve (APR) based on the estimated occluded myocardial perfusion and post-treatment myocardial perfusion, and selecting a treatment for the subject if the APR is greater than a threshold value.

### CROSS-REFERENCE TO RELATED APPLICATIONS

This application claims the benefit of priority from U.S. Provisional Application Ser. No. 63/337,607 filed on 2 May 2022, the content of which is incorporated by reference herein in its entirety.

### STATEMENT REGARDING FEDERALLY SPONSORED RESEARCH OR DEVELOPMENT

This invention was made with government support under EB021955 awarded by the National Institutes of Health. The government has certain rights in the invention.

### MATERIAL INCORPORATED-BY-REFERENCE

Not applicable.

### FIELD OF THE INVENTION

The present disclosure generally relates to methods and systems for generating a multi-level perfusion model of the cardiovascular system of a subject, methods of using the perfusion model for the diagnosis of a coronary artery stenosis, and methods of using the perfusion model for the selection of a treatment.

### BACKGROUND OF THE INVENTION

Fractional flow reserve (FFR) is the current gold standard metric for grading coronary artery stenoses based on the FAME and FAME 2 trials. However, results of the recent ISCHEMIA trial which used FFR to help guide treatment decisions demonstrate no significant improvement in primary outcomes in patients treated with percutaneous coronary intervention (PCI) with optimal medical therapy (OMT) versus OMT alone. This indicates that FFR applied according to current best-practice guidelines may be inadequate for stratifying patients with stable ischemia into treatment groups. One reason may be that microvascular disease is a potential confounding factor in the FFR assessment of epicardial coronary stenosis.

Measurement of FFR by pressure-wire invokes multiple simplifying assumptions, many of which are contradictory to known physiological properties of the coronary arterial system (e.g. steady rather than pulsatile flow, rigid rather than elastic/collapsible vessel walls, and absence of ventriculo-vascular interactions). Moreover, FFR provides only a fractional estimate of flow deficit attributable to a stenosis averaged over the entire perfusion territory supplied by an epicardial artery and provides no information on either absolute or relative perfusion deficit on a spatial basis within that territory. This spatial information is key to identifying myocardial regions of ischemic vulnerability provoked by a stenosis and for predicting the degree to which these vulnerable regions can be reversed by therapy, both invasive and medical.

### SUMMARY OF THE INVENTION

Among the various aspects of the present disclosure is the provision of methods and systems for selecting a treatment for a subject with an epicardial stenosis.

In one aspect, a computer-implemented method for selecting a treatment for a subject with an epicardial stenosis is disclosed that includes providing a multi-level perfusion model configured to estimate a myocardial perfusion for the subject. The multi-level perfusion model includes a 3D epicardial mesh representative of an aortic root, left and right coronary arteries, and associated epicardial branches of the subject, and further includes the epicardial stenosis. The method further includes estimating an occluded myocardial perfusion using the multi-level perfusion model, modifying the 3D epicardial mesh to represent the treatment of the stenosis, estimating a post-treatment myocardial perfusion using the modified multi-level cardiac perfusion model, estimating an absolute perfusion reserve (APR) based on the estimated occluded myocardial perfusion and post-treatment myocardial perfusion, and selecting a treatment for the subject if the APR is greater than a threshold value. In some aspects, absolute perfusion reserve (APR) is estimated according to the equation:

$$APR = ( {\sum }_{v ∈ R} \overline{Q} ( v ) m ( v ) \vert {Ω}_{e 1 , N} - {\sum }_{v ∈ R} \overline{Q} ( v ) m ( v ) \vert {Ω}_{e 1 , S} ) / {\sum }_{v ∈ R} m ( v )$$

wherein Σ_{v∈R}Q̅(v)m(v)|Ω_{e1,N} and Σ_{v∈R}Q̅(v)m(v)|Ω_{e1,S} represent the estimated post-treatment and occluded myocardial perfusions, respectively, v∈R represents a voxel v within a region R of a myocardium, Q̅(v) represents an average perfusion within the voxel v, m (v) represents a myocardial mass of voxel v, and Ω_{e1,S} and Ω_{e1,N} correspond to the 3D meshes with the stenosis and with the stent, respectively, during maximum hyperemia. In some aspects, the multi-level perfusion model further includes a coronary microcirculation model configured to estimate the myocardial perfusion. The coronary microcirculation model is operatively coupled to the 3D epicardial mesh at a plurality of epicardial outflow nodes defining a myocardial perfusion region and the coronary microcirculation model includes a spatially-lumped model representative of average or layer-wise perfusion over each myocardial perfusion region or a finite element mesh representative of voxel-wise perfusion over each myocardial perfusion region. In some aspects, the multi-level perfusion model further includes a varying-elastance heart model operatively coupled to the aortic root representation of the 3D epicardial mesh. The varying-elastance heart model is configured to provide an elastance driving function to drive a representation of pulsatile blood pressure and flow within the multi-level perfusion model. In some aspects, the multi-level perfusion model further includes a lumped-compartment model of peripheral circulation operatively coupled to the aortic root representation of the 3D epicardial mesh. The lumped-compartment model of peripheral circulation is configured to represent systemic and pulmonary resistances, compliances, and inertances used to drive the representation of pulsatile blood pressure and flow within the multi-level perfusion model. In some aspects, the multi-level perfusion model further includes a Voronoi model configured to define the myocardial perfusion regions. In some aspects, the method further includes producing the multi-level perfusion model based on medical imaging data, the medical imaging data comprising cCTA data, MRI data, PET perfusion data, and any combination thereof. In some aspects, producing the multi-level perfusion model further comprises receiving cCTA data, segmenting the cCTA data to produce a 3D representation of the epicardial blood vessels, and transforming the 3D representation of the epicardial blood vessels into the 3D epicardial mesh. In some aspects, producing the multi-level perfusion model further includes receiving cCTA data and transforming the cCTA data into a 3D epicardial mesh using a machine learning model. In some aspects, the machine learning model includes a convolutional neural network. In some aspects, the convolutional neural network is a graph convolutional network. In some aspects, producing the multi-level perfusion model further includes receiving the PET perfusion data and defining a plurality of perfusion parameters defining the coronary microcirculation model to match the PET perfusion data. In some aspects, defining the plurality of perfusion parameters defining the coronary microcirculation model to match the PET perfusion data further includes assigning an initial set of perfusion parameters to an AHA 17-segment model and refining the initial set of perfusion parameters of the AHA 17-segment model using Voronoi partitioning with weighted Voronoi diagrams to account for patient-specific epicardial coronary anatomy. In some aspects, defining the plurality of perfusion parameters to match the PET perfusion data further includes receiving cCTA data and PET perfusion data and transforming the cCTA data and PET perfusion data into a detailed model of the microcirculatory network using space-filling fractals and constrained constructive optimization. In some aspects, modifying the 3D epicardial mesh to represent the treatment of the stenosis includes replacing a region of the 3D epicardial mesh representative of the stenosis with a substitute region representative of the region with blockage associated with the stenosis removed or the region modified to represent an implanted stent. In some aspects, the method further includes modifying the 3D epicardial mesh to represent an implantation of a first stent as a first treatment of the stenosis, estimating a first post-treatment myocardial perfusion using the modified multi-level cardiac perfusion model, modifying the 3D epicardial mesh to represent an implantation of a second stent as a second treatment of the stenosis, estimating a second post-treatment myocardial perfusion using the modified multi-level cardiac perfusion model, estimating a first absolute perfusion reserve (APR) based on the estimated occluded myocardial perfusion and first post-treatment myocardial perfusion, estimating a second absolute perfusion reserve (APR) based on the estimated occluded myocardial perfusion and second post-treatment myocardial perfusion, and selecting a treatment for the subject associated with the higher of the first and second absolute perfusion reserves (APRs).

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5A shows images from step 1 of the workflow for semi-automated coronary mesh generation from cCTA where coronary segmentation using 3D Slicer is performed. Calcifications in the LAD (circle) are excluded.

FIG. 5B shows images from step 2 of the workflow for semi-automated coronary mesh generation from cCTA where surface mesh generation and post-processing using VMTK are performed. Post-processing steps include centerline generation, boundary patch generation, and surface patching. Shading corresponds to the distance (in mm) of the surface to the centerline, and each surface patch is assigned a minimum centerline distance used in mesh refinement in FIG. 5C.

FIG. 5C shows images from step 3 of the workflow for semi-automated coronary mesh generation from cCTA where 3D volumetric mesh generation using cfMesh is performed. Note that the algorithm automatically refines the mesh in proportion to the centerline distance such that smaller vessels have a finer mesh. Also note the presence of boundary layers (arrow), an import feature for CFD.

FIG. 6A is a schematic of the generalized model accounting for patient-specific coronary topology. Lines, circles, shaded areas, and dash lines represent epicardial arteries, outflow nodes, perfusion territories, and microvascular collaterals.

FIG. 6B is a schematic of the microcirculatory model accounting for ventricular-vascular interactions.

FIG. 6C is a graph of exemplary simulations of the pulsatile flow of the subepicardium and subendocardium.

FIG. 6D is a graph of transmural flow distribution in response to maximal vasodilation of the subepicardium and subendocardium.

FIG. 7 is a table that summarizes the models used in the data analysis workflow and the data informing each model.

FIG. 8A is a schematic illustration of a linear resistive network model of a 2-vessel coronary circulatory system, where Pₐ represents aortic pressure, Pᵥ central venous pressure, P_{d1} and P_{d2} the distal epicardial (or pre-arteriolar) pressures of vessels 1 and 2, respectively, and each Rₓ and Fₓ representing the resistances and flows associated with the epicardial (e1 and e2) components (outlined by top dotted box) and microcirculatory (m1 and m2) and collateral (c) components (outlined by bottom dotted box) of the network.

FIG. 8B is a schematic of an equivalent source-load representation of the linear resistive network model of FIG. 8A.

FIG. 8C is a graph of myocardial flow Fₘ₁ normalized to source pressure Pₛ as a function of microvascular conductance Gₘ₁, expressed as Fₘ₁/Pₛ=Gₘ₁/(1+Gₘ₁/Gₛ), given a source conductance Gₛ of 1000, 30, 10, 4, 1, and 0.1 (top to bottom).

FIG. 8D is a graph of the relationship between distal epicardial pressure P_{d1} normalized to Pₛ as a function of Gₘ₁, expressed as (P_{d1}−Pᵥ)/Pₛ=1/(1+Gₘ₁/Gₛ) over the range of Gₛ shown in FIG. 8C.

FIG. 9A is a schematic illustration of an AHA 17-segment model of cardiac perfusion distribution. This diagram illustrates the different patterns of perfusion that can be simulated with the 3 different approaches for modeling perfusion distribution. These approaches include a spatially lumped model resulting in the pattern illustrated in the basal anteroseptal wall, a spatially lumped layered model resulting in the pattern illustrated in the basal anterior wall, and a spatially detailed model which can account for perfusion gradients within segments as shown in the inferior walls.

FIG. 9B is a schematic of a varying elastance model of the heart coupled to lumped parameter models of the peripheral circulation.

FIG. 10A is a graph showing θ(t) of a varying elastance driver function used in a varying elastance heart model and lumped parameter peripheral circulatory model used to simulate whole-body hemodynamics. The model was simulated for 60 seconds to achieve a periodic steady state which required ˜1 second of compute time.

FIG. 10B is a graph showing e(t) of the varying elastance driver function used in a varying elastance heart model along with the θ(t) of FIG. 10A and lumped parameter peripheral circulatory model used to simulate whole-body hemodynamics.

FIG. 10C is a graph of the volume of the septal free wall compartment (Vₛₚₜ) of the lumped parameter peripheral circulatory model as driven by the varying elastance heart model of FIGS. 10A and 10B.

FIG. 10D is a set of graphs showing the volumes of the left and right ventricle compartments (Vₗᵥ and Vᵣᵥ) of the lumped parameter peripheral circulatory model as driven by the varying elastance heart model of FIGS. 10A and 10B.

FIG. 10E is a set of graphs showing the pressures of various compartments of the left and right ventricle Pₐₒ, Pₗᵥ, Pₚᵤ, Pₚₐ, Pᵣᵥ, and P_{vc}) of the lumped parameter peripheral circulatory model as driven by the varying elastance heart model of FIGS. 10A and 10B.

FIG. 10F is a set of graphs showing the volumes of the left and right ventricle compartments (Vₗᵥ, Vᵣᵥ Vₐₒ, Vₚᵤ, Vₚₐ, and V_{vc}) of the lumped parameter peripheral circulatory model as driven by the varying elastance heart model of FIGS. 10A and 10B.

FIG. 10G is a set of graphs showing the flow volume of the left and right ventricle compartments (Q_{sys}, Qₘₜ Qₐᵥ, Qₚᵤₗ, Qₚᵥ, and Q_{tc}) of the lumped parameter peripheral circulatory model as driven by the varying elastance heart model of FIGS. 10A and 10B.

FIG. 10H is a set of graphs showing the pressure-volume loops for the right and left ventricles of the lumped parameter peripheral circulatory model as driven by the varying elastance heart model of FIGS. 10A and 10B.

FIG. 11A is a map of the magnitude of the velocity vector field plot obtained from a simulated non-dimensionalized 3D flow over a cylinder using the incompressible Navier-Stokes solver mini-app built into the MFEM library.

FIG. 11B is a map of the pressure field from a simulated non-dimensionalized 3D flow over a cylinder using the incompressible Navier-Stokes solver mini-app built into the MFEM library.

FIG. 11C is a map of the streamlines associated with the velocity vector field from a simulated non-dimensionalized 3D flow over a cylinder using the incompressible Navier-Stokes solver mini-app built into the MFEM library.

FIG. 12A is a map of the temperature field simulated on a block finite element domain representing the soil at variable depth below the Earth's surface.

FIG. 12B is a graph of the simulated temperature at the Earth's surface. The lines correspond to the simulated temperature and the corresponding exact analytical solution. On the surface, the lines overlap since the temperature on the surface is prescribed as a Dirichlet boundary condition.

FIG. 12C is a graph of the simulated temperature at a depth 4 blocks above the lowest depth. The guess provided for the initial condition results in an initial transient that requires approximately 50 cycles to decay to a periodic steady state.

FIG. 12D is a graph of the simulated temperature at a depth 4 blocks above the lowest depth obtained using the same initial guess as used for FIG. 12C provided to the shooting operator which converged to a periodic steady state after only 3 iterations of the shooting method outer Newton solver. The converged solution was subsequently simulated for 50 more cycles showing that the solution remains at the periodic steady state throughout this time.

FIG. 13A contains images from a PET perfusion dataset acquired as a research examination simultaneously with the coronary CT examination shown in FIG. 5. As shown in FIG. 5, CTA revealed approximately 50% stenosis in the mid-LAD region. However, myocardial perfusion reserve (MPR), defined as the fraction of perfusion in the LAD region during stress over perfusion at rest, was within normal limits. The patient was subsequently taken for catheter angiography, and iFR performed at the site of stenosis in the LAD measured 0.9 corresponding to a normal value. FFR_{CT} was not performed on this patient as HeartFlow was not available at the institution at the time of the scan.

FIG. 13B is a set of perfusion and flow maps obtained from the PET perfusion dataset of FIG. 13A.

FIG. 13C is a table of summary parameters obtained from the PET perfusion dataset of FIG. 13A.

FIG. 13D contains a pair of graphs showing time activity curves obtained from the PET perfusion dataset of FIG. 13A.

FIG. 14 is a table of physiological indices (CFR, FFR, MPR, FPR, APR) related to blood flow and perfusion.

FIG. 15 is a table comparing the capabilities of the physiological indices in FIG. 14 related to the key aspects of the disclosed physiological index.

FIG. 16 is a set of spatial maps of APR in an occluded artery as measured pre-PCI and as predicted post-PCI.

FIG. 17A contains myocardial PET perfusion images and corresponding maps of perfusion obtained during rest and stress, and analogous maps for pre- and post-treatment conditions.

FIG. 17B is a table of MPR values showing the MPR (“CFR”) for each vessel segment based on the myocardial PET perfusion images and corresponding maps of perfusion of FIG. 17A.

FIG. 17C a table and corresponding maps of flow and myocardial perfusion reserve (MPR) values for various vessel segments based on the myocardial PET perfusion images and corresponding maps of perfusion of FIG. 17A.

### DETAILED DESCRIPTION OF THE INVENTION

The present disclosure is based, at least in part, on the discovery that the image analysis described herein provides a quantitative value (absolute perfusion reserve; APR) that provides more accurate measurements for grading coronary artery stenosis than current gold standards. As described herein, a workflow is developed to quantify an APR value on an individualized patient basis to inform subsequent interventions in patients with coronary artery stenosis.

In the present disclosure, a new non-invasive method to quantify the physiological significance of a coronary artery stenosis is described. The method derives physiologically based patient-specific models of both large vessel epicardial flow dynamics and myocardial microvascular perfusion using combined imaging data from coronary artery CTA and myocardial perfusion (which can come from either PET, dynamic contrast-enhanced CT, or MRI perfusion studies). Flow dynamics of the aortic root and epicardial coronary arteries are described using 3D computational fluid dynamics models based on the Navier-Stokes equations. Boundary conditions to the aortic inflow and outflow are provided by a varying-elastance type model of cardiac mechanics and lumped compartment type models of the peripheral and pulmonary circulation, respectively. Distributed 1D and or lumped compartment models of the coronary microcirculation serve both to provide boundary conditions to the epicardial outflow branches and to characterize microvascular flow dynamics and myocardial perfusion. The models can be personalized to individual patient anatomy and physiology, with parameters of the models derived directly from the anatomic information provided by gated cardiac CT and functional perfusion information provided by myocardial perfusion imaging that is acquired contemporaneously.

In some aspects, myocardial perfusion and alterations in perfusion due to epicardial stenosis can depend critically on parameters of the microcirculation (including microvascular density, distribution, resistance, etc.) and these parameters can vary both spatially within the myocardium and between patients. In some aspects, the method includes developing a multi-scale modeling framework using CFD for epicardial hemodynamics and myocardial perfusion including pulsatile effects and ventricular-vascular interactions. In some embodiments, data provided by coronary CTA (cCTA) and PET perfusion can be rich enough to alone inform the physiological model parameters. In some embodiments, additional imaging/clinical data or assumptions can be included in the model. In some embodiments, the accuracy of the model is validated by comparing model predictions to data obtained from independent pressure waveform measurements obtained by sensor wire measurements during coronary catheterization on the same patient.

In some aspects, model development and implementation can include multiscale multiphysics modeling of coronary hemodynamics. In some embodiments, models of coronary hemodynamics can include 3D computational fluid dynamics (CFD) models of the aortic root and epicardial coronary arteries based on the incompressible Navier-Stokes (NS) equations, which can be coupled to the OD ordinary differential equation (ODE) lumped parameter models of ventricular mechanics and systemic and pulmonary hemodynamics. In some embodiments, the coupling of the 3D CFD model to the OD ODE models can occur at the inflow and outflow boundaries of the 3D domain and can use a fully implicit Newton linearization scheme. In some embodiments, for the CFD component of the model, fast preconditioned Newton-Krylov matrix-free methods can be used to increase memory efficiency in combination with multigrid parallelization methods to significantly reduce computational time. In some embodiments, to improve computational time and efficiency, adaptive mesh refinement and shooting methods for parallel in time numerical integration can be integrated into the method.

In some embodiments, a patient can have concurrent PET perfusion and coronary CTA data, as well as angiographic data and pressure wire measurements from coronary catheterization. In some embodiments, a semi-automated approach for segmenting the epicardial coronary arteries from coronary CTA for use in the computational fluid dynamics models, as well as for segmenting the myocardium for the perfusion component of the model, can be used. In some embodiments, the software MIM Encore can be used to extract quantitative perfusion data from the PET perfusion images. In some embodiments, an automated method for segmentation based on machine learning can be used.

A unique feature of the technology is the incorporation of perfusion data in the modeling analysis which allows direct parameterization of the microvascular components of the model without requiring assumptions about the microvascular properties that are required in existing commercial technologies such as FFR-CT (HeartFlow). Because of this requirement for assumptions regarding the microcirculation, existing technologies are not able to account for the presence and severity of microvascular disease in their analysis of large vessel flow dynamics. Moreover, those technologies are not able to diagnose a microvascular disease or quantify its severity independently of the presence of large vessel disease. With this technology, variation between patients and within different regions of myocardium in microvascular function can be accounted for and microvascular disease on a patient-specific basis can be directly quantified, even in patients with otherwise normal large vessel function. It is anticipated that better characterization of the microcirculation using the described methods will improve the accuracy of non-invasive FFR prediction compared to existing technologies, particularly in cases of borderline FFR where FFR-CT was previously demonstrated to yield relatively low-accuracy predictions.

Beyond this improvement in existing technologies, the technology enables an entirely new metric by which to grade the physiological significance of a coronary artery stenosis called absolute perfusion reserve. This metric quantifies, in absolute value (i.e. blood perfusion in mL/min per mg myocardial tissue), the degree of perfusion deficit that can be attributed to a large vessel stenosis as well as the degree of perfusion improvement that can be expected to result from interventions such as coronary artery stenting. This addresses two major limitations associated with FFR, which are its inability to account for the spatial distribution within the myocardium of flow supplied by an epicardial artery and the inability to quantify flow deficit in absolute terms. These details are necessary to be able to predict how perfusion can be expected to change with intervention. Ultimately, the severity of coronary ischemia is directly related to the severity of perfusion deficit, and the predictive ability of any test designed to predict response to intervention requires accurate prediction of perfusion response.

As described above, the technology allows the prediction of perfusion response to intervention which allows for a new clinical test that is fundamentally different from currently available testing paradigms. The current gold standard metric for determining whether or not to perform an intervention on a coronary artery stenosis is fractional flow reserve, an invasive measurement that is made in the cardiac catheterization lab. FFR-CT, a currently available commercial technology, allows a non-invasive means of predicting FFR based on CT imaging. However, a positive test still requires confirmation in a cath lab prior to proceeding with intervention. Patients with false-positive results on FFR-CT will have undergone an unnecessary invasive procedure, whereas patients with false-negative on FFR-CT could be missing out on potentially life-saving therapy. More accurate prediction of FFR, which this technology allows, will reduce the number of these false-positive and false-negative cases. Perhaps of greater impact however is that our technology will allow for a superior and more predictive test than the current gold standard FFR which we call APR. This test is both non-invasive and relies only on routine imaging that is commonly performed in clinical practice.

In various aspects. APR is defined as:

$$APR = ( {\sum }_{v ∈ R} \overline{Q} ( v ) m ( v ) \vert {Ω}_{e 1 , N} - {\sum }_{v ∈ R} \overline{Q} ( v ) m ( v ) \vert {Ω}_{e 1 , S} ) / {\sum }_{v ∈ R} m ( v ) \qquad ( 1 )$$

where v∈R corresponds to an individual voxel (or finite element) in a region R of the myocardium, Q̅(v) represents average perfusion within voxel v, m(v) is the myocardial mass of voxel v, and Ω_{e1,S} and Ω_{e1,N} correspond to the coronary mesh in the case that epicardial artery e1 has a stenosis present and the mesh following treatment of the stenosis, respectively, during maximum hyperemia.

According to this definition, APR can be defined for individual voxels which allows APR maps to be generated voxel-wise for the whole heart. Alternatively, APR can be defined for an individual region R of the myocardium which provides for some flexibility. For example, R may correspond to the perfusion territory of an individual coronary artery, and these territories could be further subdivided into subepicardial, mid-myocardial, and subendocardial regions.

Several derivatives of this definition can also be considered as alternative biomarkers. For example, fractional perfusion reserve (FPR) may be used as a perfusion biomarker analogous to FFR. FPR is defined as:

$$FPR = {\sum }_{v ∈ R} \overline{Q} ( v ) m ( v ) \vert {Ω}_{e 1 , S} / {\sum }_{v ∈ R} \overline{Q} ( v ) m ( v ) \vert {Ω}_{e 1 , N} \qquad ( 2 )$$

In the case that R corresponds to the coronary artery distribution of epicardial artery e1, this would be identical to FFR for e1 assuming there is no spatial overlap of microvascular territories. Note though that this would represent true FFR and not necessarily FFR as estimated by pressure wire or FFR_{CT}.

One aspect of the disclosure is the use of multiscale models. In some embodiments, part of our multiscale model includes a finite element mesh of the myocardium. The mesh elements can correspond to voxels from a perfusion scan or can be more coarse or fine in granularity. In some embodiments, the multiscale models explicitly model the microcirculation and element-wise perfusion is a model variable. In some embodiments, the model can directly simulate perfusion maps that look identical to those provided e.g. by a PET perfusion scan, and parameters of our model can be estimated using data from these perfusion scans.

In some embodiments, determining APR using the model is a two-step process. In these embodiments, the first step is the inverse problem of estimating model parameters from the imaging data. The next is the forward problem of simulating perfusion maps after adjusting the epicardial coronary mesh to reflect the removal or other treatment of stenosis. The simulated maps provide the first term in the numerator in Eq. (1) and the measured maps obtained from perfusion imaging provide the second term in the numerator. In some embodiments, the method provides APR values for each major coronary artery branch, as well as the subepicardial, mid-myocardial, and subendocardial segments of each perfusion territory. In other embodiments, the method allows for a clinician to manually select a set of voxels using a selection tool and quantify an APR value corresponding to that voxel set. One aspect of the method is the segmentation of vessels from the images. In some embodiments, the method allows a clinician or a radiologist the ability to fine-tune the segmentation. In some embodiments, the method provides automated mesh generation that clinicians can evaluate and correct if needed. In some embodiments, the method provides differential perfusion maps. In some embodiments, the method provides absolute perfusion maps. In some embodiments, the method provides fractional perfusion maps. In some embodiments, the method provides APR spatial maps. In some embodiments, the method provides any combination of absolute perfusion maps, differential perfusion maps, fractional differential maps, or APR spatial maps.

In some embodiments, the method allows the prediction of perfusion following virtual stent placement. This virtual treatment response can be specific to an individual patient's anatomy and physiology that is defined from the imaging data. In one embodiment, the method provides a tool/software for clinicians to test various stent sizes/configurations to determine how perfusion would change. In one particular embodiment, the patients can have chronic total occlusion of an epicardial vessel without ischemic changes when perfusion is supplied via collaterals from a patent branch. In another embodiment, mild stenosis in a patient can be functionally significant when it is supplying multiple collaterals to adjacent vessels. In another embodiment, APR can be measured on a chronically occluded vessel. In various embodiments, the method can be performed on multiple vessels. In some of these embodiments, the method is performed before a cath lab procedure. In some embodiments, the method helps plan a future procedure that includes but is not limited to the introduction of a stent.

In some embodiments, a threshold or cutoff range of APR detects treatable ischemia and predicts outcomes. In some embodiments, a patient with an APR value below the threshold is treated. In some of these embodiments, a patient with an APR value below the threshold is treated with the introduction of a stent. In some embodiments, this APR threshold value is from about 0.7 to about 0.9. In some embodiments, a patient with an FPR value below the threshold is treated. In some of these embodiments, a patient with an FPR value below the threshold is treated with the introduction of a stent. In some embodiments, this FPR threshold value is between 0.7-0.9. In some embodiments, APR or FPR is defined for a specific coronary artery perfusion territory. In some embodiments, APR or FPR is defined within the subendocardial zone of a perfusion territory. In some embodiments, APR and FPR are defined within the subendocardial zone or the subepicardial zone. In some embodiments, APR or FPR within the subendocardial and subepicardial zones is compared. In some embodiments, the ratio between the APR or FPR values within the subendocardial zone relative to the subepicardial zone detects treatable ischemia and predicts outcomes. In some embodiments, a threshold value of this ratio detects treatable ischemia and predicts outcomes. In some of these embodiments, a patient with a ratio above or below the threshold is treated with the introduction of a stent.

In some embodiments, the system of the present disclosure can automatically detect the most severe anatomic stenosis (based e.g. on either maximum percent luminal stenosis or minimum FFR) and then calculate APR based on the treatment of this stenosis. In some embodiments, if APR is within some threshold, the system can recommend stent placement or not. In some embodiments, the system can also calculate a separate APR based on the normalization of microvascular parameters. In some embodiments, this information can be used in tandem when developing the recommendation. In some embodiments, if stent-based APR is high and microvascular APR low, then the system can recommend stent placement. In some embodiments, if stent APR is low and microvascular APR high, then the system can recommend medical therapy alone. In some embodiments, a ratio or other derived quantity of these indices can be of value. In some embodiments, the index most relevant to the outcome is determined. In some embodiments, the model can optimize that index to provide the best recommendation.

In some embodiments, the method provides fractional flow reserve (FFR) values. In some embodiments, FFR is defined as the ratio of the pressure at a point along a coronary artery to the aortic pressure. In some embodiments, pulsatile models are included in the method. In some embodiments, patients are selected for treatment based on a threshold FFR value. In some embodiments, the threshold or cutoff FFR is between 0.7 and 0.9. In some embodiments, a patient with FFR values below the threshold FFR value is subsequently treated. In some embodiments, this treatment is the introduction of a stent in a patient.

One aspect of this disclosure is a method of analysis to quantify APR, FPR, FFR, or other related parameters. In some embodiments, this includes the acquisition of a 3D image. In some embodiments, the 3D image can be but is not limited to a CT x-ray, PET, or MRI image. In some embodiments, more than one image type from a single patient is analyzed with the method. In one particular embodiment, CT and PET images are both used in the analysis.

Another aspect of the method includes the segmentation of the coronary artery from the 3D image. In some embodiments, segmentation occurs automatically. In some embodiments, segmentation occurs with semi-automated threshold-based methods. In some embodiments, segmentation occurs with the manual tracing of vessels. In some embodiments, coronary segmentation is performed using the voxel-based tools available in 3D Slicer. In some embodiments, machine learning (ML) and convolutional neural networks (CNNs) are applied in coronary segmentation. In some embodiments, methods incorporating shape prior information are employed; this approach can exploit the tubular shape of vessel segments along with deep learning methods to deform the tubular geometry to match the wall of the lumen in cCTA images. In some embodiments, the ML allows for sub-voxel accuracy and direct generation of a smooth surface mesh without additional processing as is usually required in voxel-based methods. In some embodiments, patient-specific 3D epicardial anatomic models will be generated using these methods. Without being limited to any particular theory, the accuracy of ML-generated models is thought to be superior to semi-automated voxel-based models, and the sensitivity of the ML-generated models to imaging artifacts is less compared to semi-automated voxel-based models.

In some embodiments, graph convolutional networks (GCNs) are used to predict the spatial location of vertices in a tubular surface mesh conforming to the coronary artery lumen. In some embodiments, this involves cCTA imaging data as well as the vessel centerlines as input. In some embodiments, the model is applied to ˜5000 cCTA datasets obtained clinically at MIR, of which ˜80% will be used for training and ˜20% for validation. In some embodiments, subsequent catheter angiography data will be incorporated into the workflow. In some embodiments, the incorporation of catheter angiography data provides for enhanced training of the GCN based on imaging features from both cCTA and angiography. The angiographic images can also serve as ground truth datasets for model testing. In some embodiments, any other suitable machine learning models and methods may be utilized using the dataset without limitation.

In various other aspects, the method further includes surface mesh generation. In some embodiments, the segmented data is post-processed to generate a surface mesh. In some embodiments, the surface meshes are processed in VMTK, an open-source vascular modeling library. In some embodiments, VMTK is used to automatically generate centerlines and to create boundary and surface patches. In some embodiments, the generated centerlines and boundary and surface patches are used to assign boundary conditions for the CFD models and in the mesh refinement algorithm used to create a 3D volumetric mesh. In some embodiments, each surface patch is associated with a minimum distance to the centerline which is used to assign a maximum cell size to each individual vessel segment during volume mesh generation.

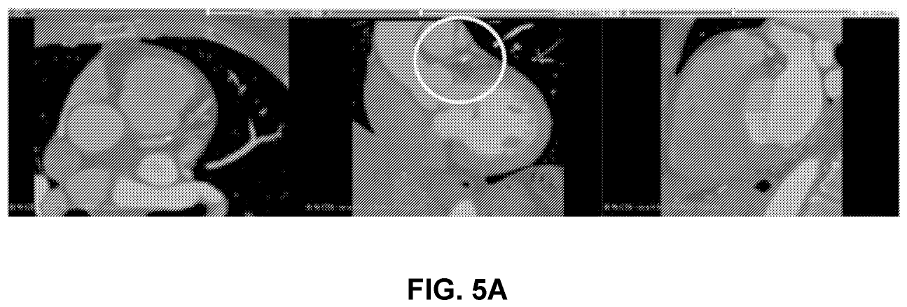

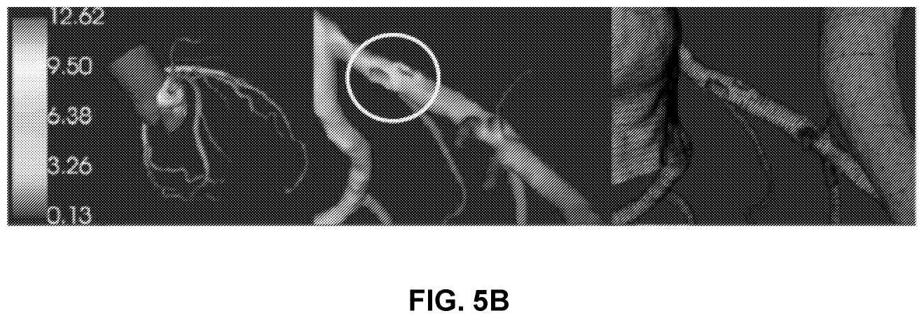

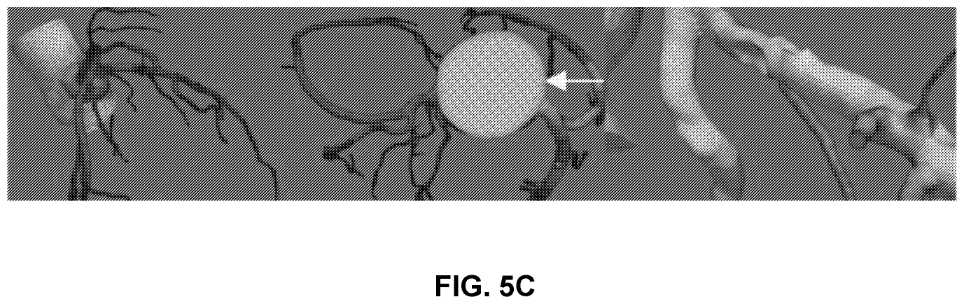

In various other aspects, the method further includes 3D volumetric mesh generation. In some embodiments, 3D volume mesh is generated using the software cfMesh. In some embodiments, the parameters required by cfMesh are obtained automatically from the processing done in the surface mesh generation methods as described above. In some embodiments, the 3D volumetric mesh generation is fully automated. An exemplary mesh from one of the cCTA datasets for which we also have PET and catheterization data is shown in FIG. 5.

A further aspect of the disclosure describes the development and use of computational flow dynamics (CFD) models. In some embodiments, thresholding, filtering, or smoothing functions are used to segment the epicardial artery lumens. In some embodiments, the CFD models make use of the 3D volumetric mesh derived from the 3D image as described above. In some embodiments, the accuracy and reproducibility of the CFD models depend on the accuracy of coronary segmentation and mesh generation. In some embodiments, thorough testing and refinement of these algorithms using clinically realistic datasets is performed. In some embodiments, the methods described herein build, identify, and validate patient-specific models of epicardial coronary hemodynamics and myocardial perfusion accounting for pulsatile flow, ventricular-vascular interactions, and individual patient anatomy and topology of the coronary epicardial arteries and microcirculation using quantitative imaging data. In some embodiments, the developed models explore the complex relationships between epicardial stenosis, microvascular disease, and myocardial perfusion and identify the mechanistic basis for known discordances between FFR, FFRCTA, MPR, and the newly proposed APR. In some embodiments, myocardial perfusion and perfusion response to stenosis depend critically on the parameters of the microcirculation. In various embodiments, microvascular parameters vary both spatially within the myocardium and between individual patients.

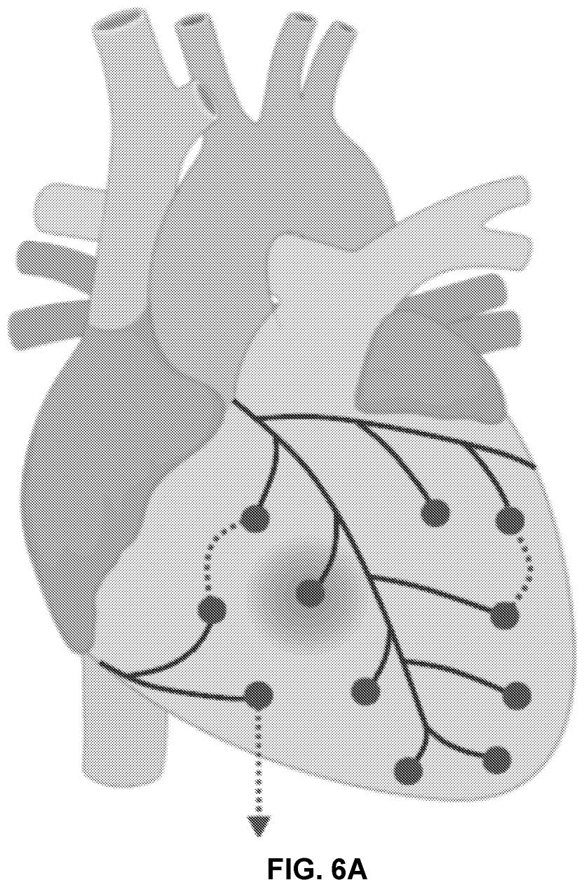

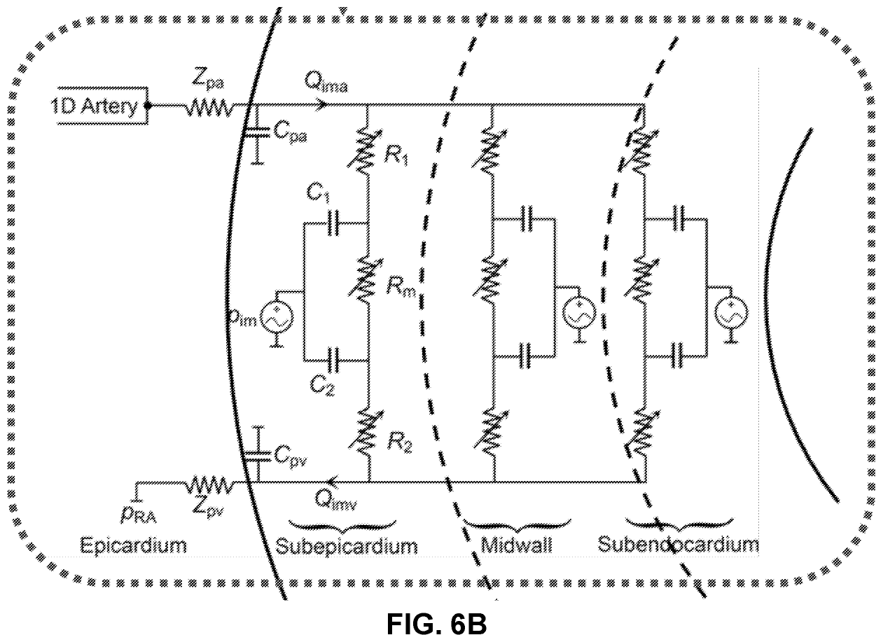

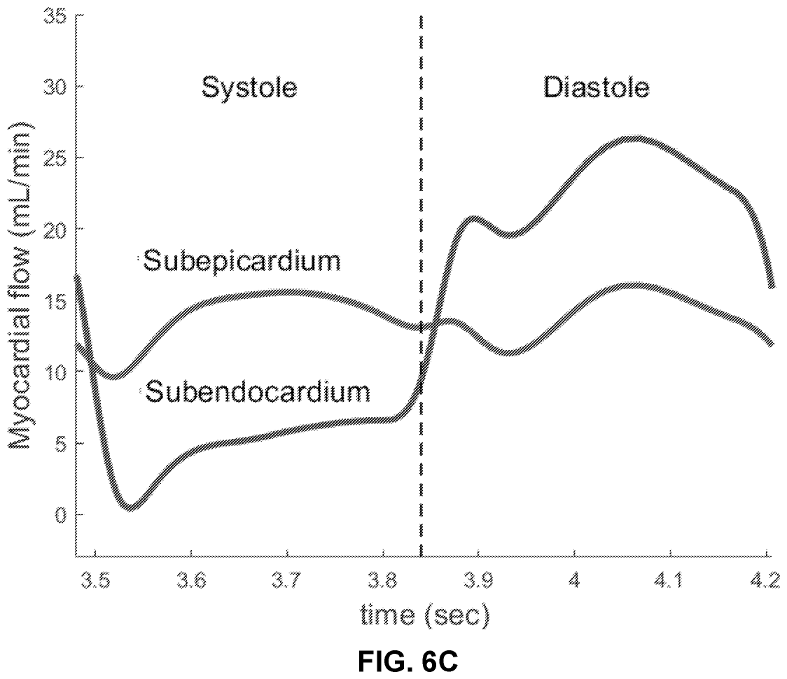

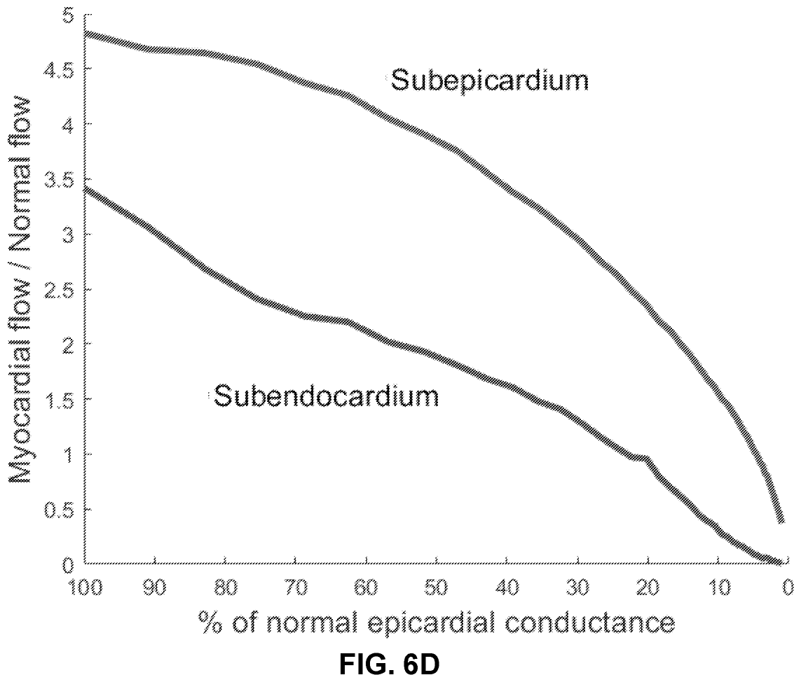

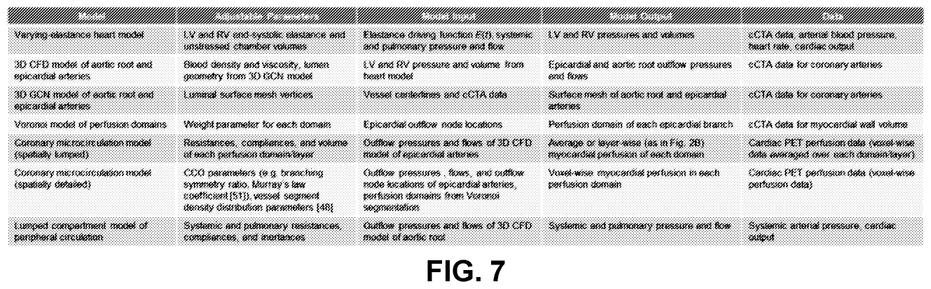

In some embodiments, the methods include the development and use of epicardial flow dynamics models. In one aspect, a general method is outlined in FIGS. 6A, 6B, 6C, and 6D, and the resulting models and data are summarized in FIG. 7. For the epicardial component, established methods can be used for modeling the epicardial arteries which generally invoke either OD lumped parameter RLC circuit representations or, in some aspects, 1 D or 3D representations based on the Navier-Stokes equations. In some embodiments, 3D models are developed to account for patient-specific coronary anatomy and complex plaque geometry. Coronary geometry can be obtained using the methods described in the Examples herein or by machine learning-based anatomical model generation as described herein.

In various aspects, the boundary conditions to the aortic inlet and outlet can be derived as done with FFR_{CTA} where inflow and outflow boundary conditions to the aortic root are provided by a varying elastance model of the heart and a lumped compartment model of the peripheral circulation, respectively, and parameters of both models are derived from cardiac output and brachial artery pressure measurements.

In yet another aspect, methods for modeling microcirculation and perfusion are disclosed. Without being limited to any particular theory, epicardial outflow boundary conditions cannot be easily measured non-invasively, and thus FFR_{CTA} estimates typically rely on either data in the literature or on a variety of heuristic assumptions such as total flow proportional to myocardial mass and microvascular resistance proportional to coronary outlet lumen cross-sectional area. In one aspect, the development of patient-specific models of microcirculation based on data from PET provides for boundary conditions to be derived directly from patient-specific data. In some embodiments, the microcirculation is represented using spatially distributed lumped networks accounting for intramyocardial pressure effects. In some aspects, the distribution of the networks can be set according to the AHA 17-segment model and refined to account for patient-specific epicardial anatomy using Voronoi segmentation with weighted Voronoi diagrams, in which smaller weights correspond to rarefaction and larger weights correspond to angiogenesis, to account for remodeling. Each segment can be further divided into 3 layers as illustrated in FIG. 6B.

In other embodiments, detailed models of microcirculatory networks are constructed. In some aspects, the topology of the microcirculation is obtained by reconstructing the microvascular networks from the CT and PET perfusion data using principles of space-filling fractals and constrained constructive optimization (CCO). In some aspects, this approach can be validated by simulating perfusion data using either actual or artificially generated networks and verifying that the reconstructed networks closely match the original networks. In some aspects, the spatially detailed over-lumped approach can account for spatial gradients and physiological flow heterogeneity within perfusion segments.

Without being limited to any particular theory, the multi-scale models described are dynamical systems characterized by large numbers of parameters representing physical entities such as resistances and compliances, and these parameters can vary greatly between patients and in disease states. In some embodiments, perfusion depends on these parameters. In some embodiments, established methods of sensitivity analysis are applied to determine sets of uncorrelated identifiable model parameters and optimization techniques including but not limited to genetic algorithms and Kalman filtering to estimate parameters from noisy cCTA and PET datasets. In one embodiment, parameters are directly manipulated in the model, and responses in perfusion are observed. In another embodiment, the Akaike Information Criterion (AIC) for different versions of models in which parameters vary on a spatial basis and between patients and where parameters are fixed are compared.

In various embodiments, the methods described herein reveal the need for additional clinical/imaging data to identify valid models. In some embodiments, model validity can be determined by comparing epicardial pressure time-series data obtained during coronary catheterization to measured waveforms. In some embodiments, post-PCI scans are obtained that enable the comparison of APR estimated from the model with APR calculated directly using data from PET obtained prior to and following invasive therapy. In some embodiments, model accuracy is scored using the Dice coefficient. In one embodiment, the Dice score of a model described herein is 0.8265±0.052. In various aspects, described in the Examples herein, the APR biomarker exhibited superior performance relative to currently available metrics such as FFR in terms of guiding decisions for invasive therapy.

In some embodiments, the models and methods disclosed herein are used to inform the treatment of stenosis of an epicardial artery. Without being limited to any particular theory, for epicardial arteries, the main effect of treatment is to alter the luminal geometry. In some embodiments, alteration of the luminal geometry is simulated in the model by modifying the epicardial artery mesh. In some embodiments, the modification can be based on the geometry of a specific stent a cardiologist plans to place. In some embodiments, an interpolation of the luminal cross-sectional areas of the undiseased segments upstream and downstream of an epicardial stenosis can be performed to estimate the effects of treatment using the models and methods disclosed herein.

In various aspects, the models and methods disclosed herein are used to inform the treatment of the microvasculature by the administration of biologically active compounds including, but not limited to, ACE inhibitors, statins, and platelet inhibitors. In some aspects, the models and methods disclosed herein are used to inform the treatment of the microvasculature either as a sole treatment or in combination with a treatment of the stenosis of an epicardial artery. In some embodiments, “normal” values for the parameters of the microcirculatory model disclosed herein can be learned. In some embodiments, this learning can occur by applying the model of the present disclosure to a cohort of healthy research patients. In some embodiments, once normal values for the parameters have been established, the effects of treatments (such as ACE inhibitors, statins, platelet inhibitors, etc.) can be predicted by adjusting the parameters to normal levels. In some embodiments, the medical therapies described in the present disclosure are effective in normalizing microcirculation. Without being limited to any particular theory, it is realistic to assume that the parameters of the microcirculatory model can be modified through medical therapy to normal levels in patients with microvascular disease. In some embodiments, applying the model to cohorts of patients can inform whether medical therapies are effective in normalizing microcirculation. In some embodiments, the parameter values can be compared in healthy patients, patients with microvascular disease, and patients with microvascular disease who have been treated with medical therapy. In some embodiments, the expected effect of the medical therapies on the parameters can be determined.

In some aspects, other parameters of the microcirculatory model can be adjusted for other types of therapies. In some embodiments, for blood pressure therapy, examples of parameters that can be adjusted are peripheral vascular resistance and total blood volume. In some embodiments, therapies such as beta-blockers can be simulated by adjusting heart rate in the model along with parameters of the ventricular elastance curve that affects diastolic relaxation and systolic contractility. In some embodiments, the effect of these therapies can be to alter the ventricular-vascular interactions responsible for a subendocardial predilection for ischemia.

In some aspects, the imaging inputs to the models and methods disclosed herein are provided in the form of dicom files for the coronary CTA and perfusion imaging studies including but not limited to, PET, CT, MRI, or other suitable imaging modalities. In some aspects, a patient's vital signs can be taken during rest and stress. In some embodiments, a patient's heart rate and blood pressure can be taken at rest and following the administration of a stress agent including, but not limited to, regadenoson. In some embodiments, recording of a patient's heart rate and blood pressure is performed as the standard protocol for perfusion imaging. In some embodiments, the models and methods described herein avoid the need to stress the patient by using resting phase images in combination with the model disclosed herein to predict stress response.

In various aspects, embodiments, the user interface can be optimized to the needs of a clinician, including but not limited to, optimizing the level of detail and control a clinician has. In some embodiments, the user interface can output an FFR number at a selected point along an artery. In some embodiments, the user interface can output an APR value for a selected vessel, which can predict the effect of stent placement in that vessel. In some embodiments, the user interface can provide a fine-detailed level of control. In some embodiments, if the patient has a multifocal disease or multivessel disease, the user interface can facilitate a clinician to interrogate the effect of treating multiple stenoses/vessels with combinations of stents. In some embodiments, the user interface can facilitate a clinician to interrogate the effect of medical therapy alone, stent alone, or a combination of medical therapy and stent(s).

In some embodiments, the user interface can assist in the choice of where to place a stent. In some embodiments, a 3D model/mesh of the epicardial arteries with superimposed heat maps encoding quantities such as percent luminal stenosis or FFR can be displayed. In some embodiments, the clinician can then choose where to place a stent based on these models, for example by choosing a segment covering the most severe anatomic stenosis. In some embodiments, the clinician can choose a proximal and distal site to correspond to the proximal and distal ends of the stent.

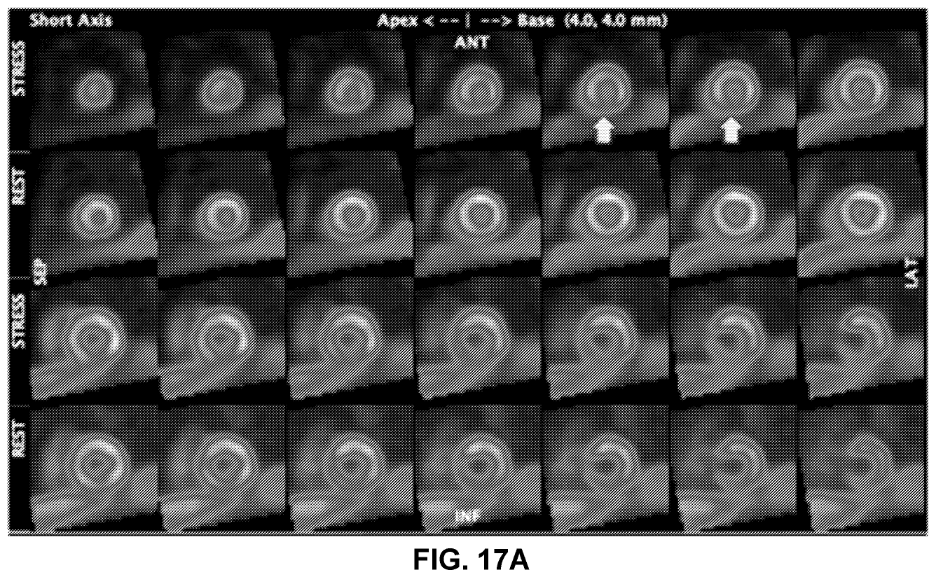

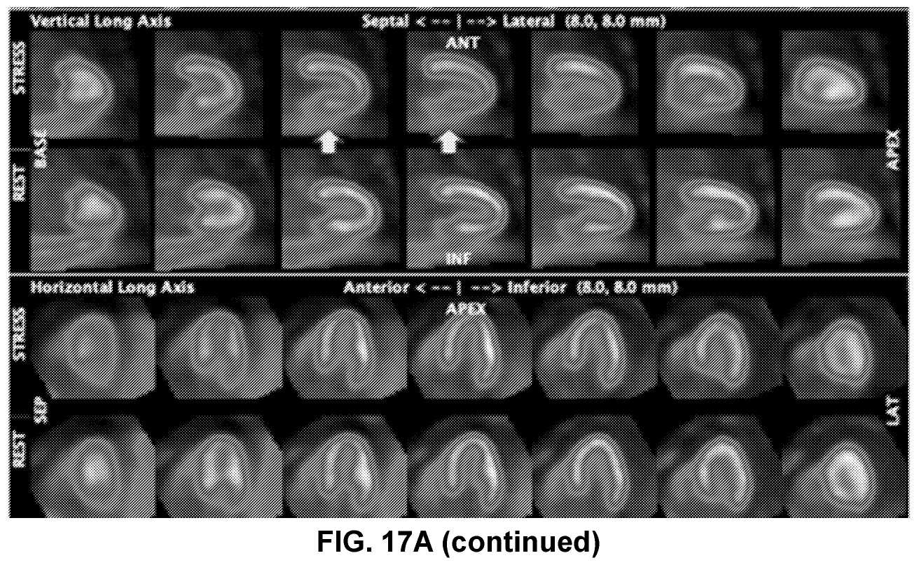

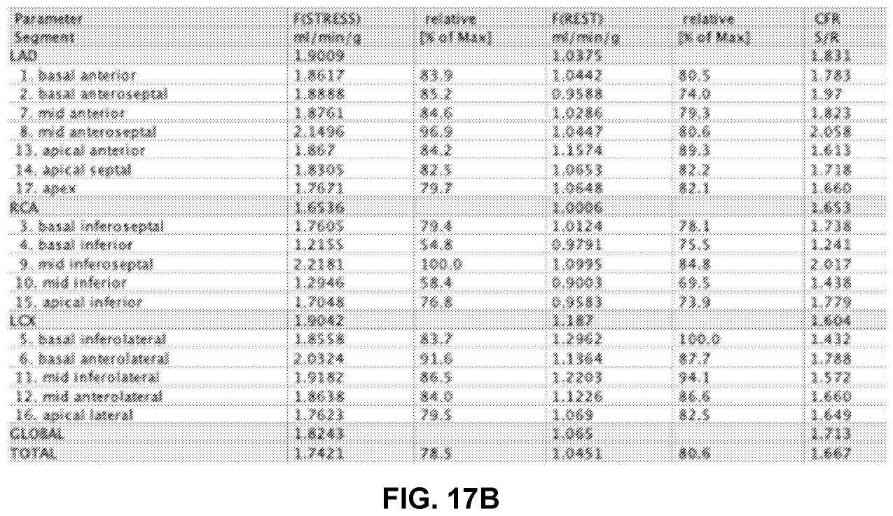

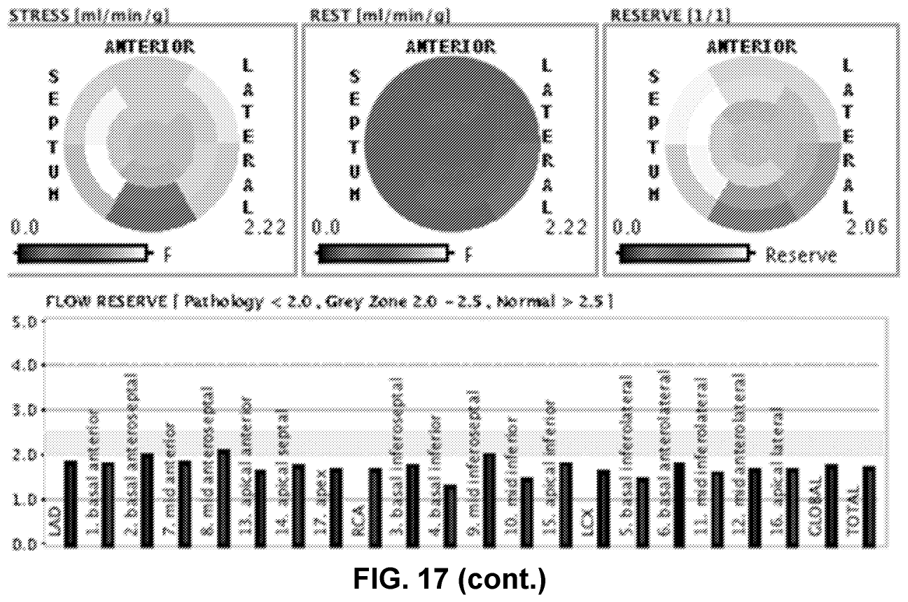

In some embodiments, a user interface is included that provides flexibility based on the needs of the clinician. In some embodiments, the results from the models and methods described herein are presented in the form of tables of data values. In other embodiments, the user interface presents the results of the models and methods described herein in the form of images or maps of myocardial perfusion pre-therapy and post-therapy, or images or maps of voxel-wise APR (difference between post-therapy and pre-therapy maps). In some embodiments, the user interface provides maps of perfusion during rest and stress, and optionally analogous maps of perfusion for pre- and post-treatment. In some embodiments, the user interface provides MPR value tables for each vessel segment. In some embodiments, the user interface provides APR values averaged over each vessel segment. In some embodiments, the user interface provides additional derived values other than voxel-wise or spatially averaged APR. Various aspects of an exemplary user interface are shown illustrated in FIG. 17. In some embodiments, the user interface provides one or more corresponding statistics of APR to a clinician including, but not limited to, maximal APR or percent APR greater than a predetermined threshold. In some embodiments, the user interface provides to the clinician in the form of 3D myocardium models or meshes with heat maps superimposed on the meshes. In some embodiments, the user interface's visualization of these meshes would allow for rotation, zooming, panning, and other features. In some embodiments, the heat maps are superimposed directly on the cross-sectional images themselves so that a clinician can scroll through the images and visually assess regions of ischemic vulnerability.

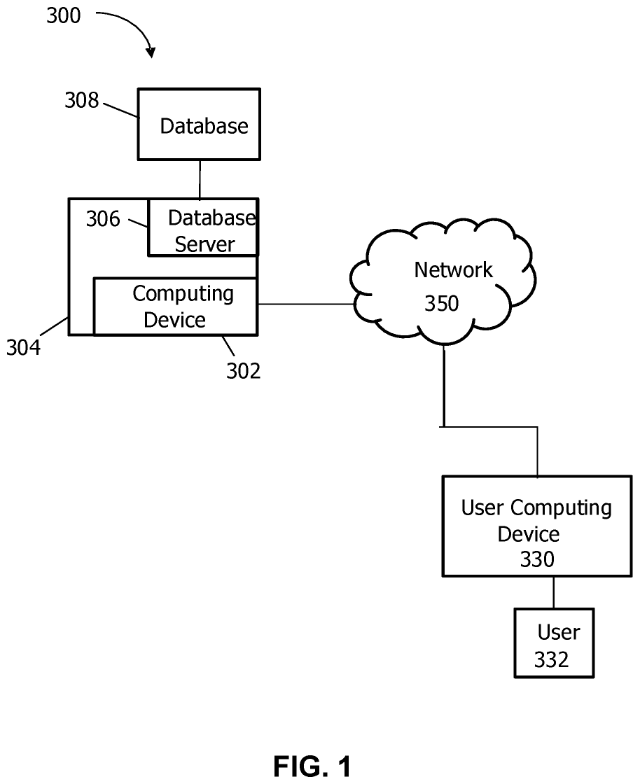

In various aspects, at least a portion of the methods disclosed herein may be implemented using various computing systems and devices as described below. FIG. 1 depicts a simplified block diagram of a computing device for implementing the image analysis methods described herein. As illustrated in FIG. 1, the computing device 300 may be configured to implement at least a portion of the tasks associated with the disclosed oscillation detection method, but not limited to producing a multi-level perfusion model based on medical imaging data of the heart of a subject including, but not limited to, CT imaging data, MRI data, or PET perfusion data. The computer system 300 may include a computing device 302. In one aspect, the computing device 302 is part of a server system 304, which also includes a database server 306. The computing device 302 is in communication with database 308 through the database server 306. The computing device 302 is communicably coupled to a user-computing device 330 through a network 350. The network 350 may be any network that allows local area or wide area communication between the devices. For example, network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

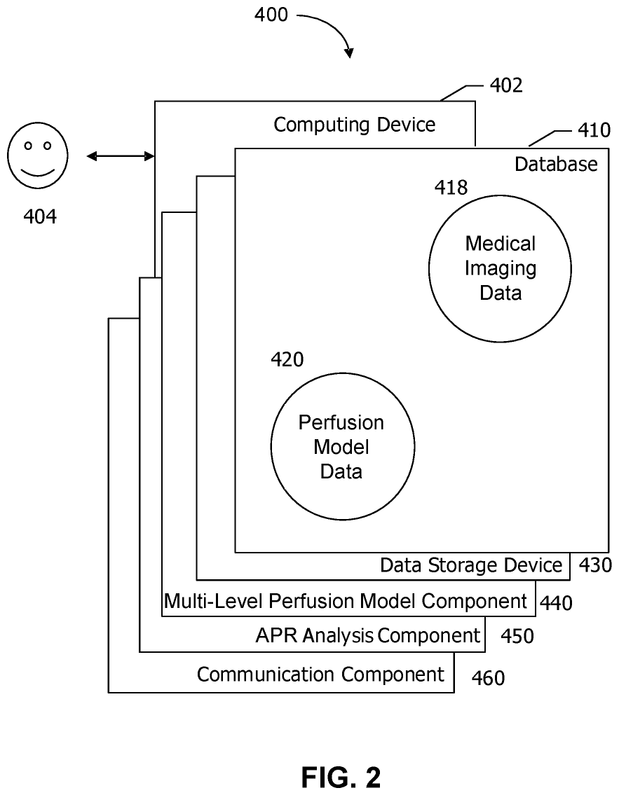

In other aspects, the computing device 302 is configured to perform a plurality of tasks associated with the production of the multi-level perfusion model and method of selecting a treatment for an epicardial stenosis using the multi-level perfusion model as described herein. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes medical imaging data 418 and perfusion model data 420. Non-limiting examples of medical imaging 418 include any data quantifying various aspects of epicardial circulation vessels and myocardial perfusion including, but not limited to, CT data such as cCTA data, MRI data, and/or PET perfusion imaging data. Non-limiting examples of suitable perfusion model data 420 include any values of parameters defining the multi-level perfusion model as described herein.

Computing device 402 also includes a number of components that perform specific tasks. In the exemplary aspect, computing device 402 includes a data storage device 430, a multi-level perfusion model component 440, an APR analysis component 450, and a communication component 460. The data storage device 430 is configured to store data received or generated by computing device 402, such as any of the data stored in database 410 or any outputs of processes implemented by any component of computing device 402. The multi-level perfusion model component 440 is configured to produce a multi-level perfusion model based on the analysis of medical imaging data as disclosed herein. The APR analysis component 450 is configured to determine absolute perfusion reserves with and without a stenosis using the multi-level perfusion model as described herein.

The communication component 460 is configured to enable communications between computing device 402 and other devices (e.g. user computing device 330 and sequencing system 310, shown in FIG. 1) over a network, such as network 350 (shown in FIG. 1), or a plurality of network connections using predefined network protocols such as TCP/IP (Transmission Control Protocol/Internet Protocol).

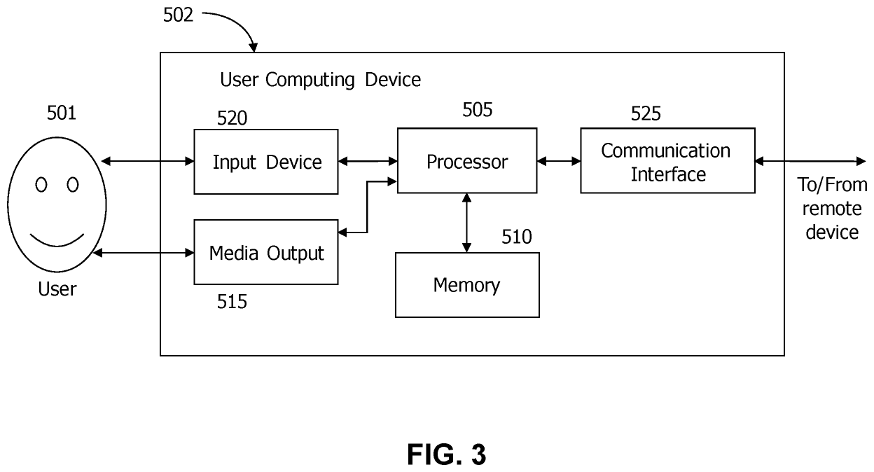

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile Communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

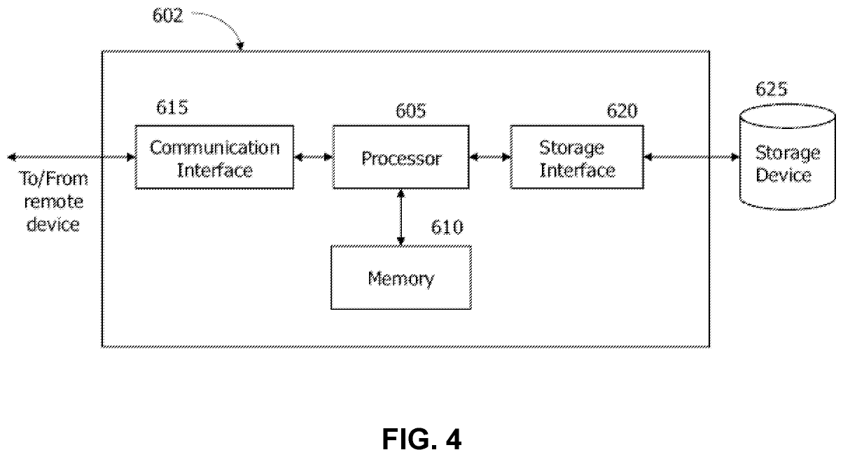

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

In various aspects, one or more machine learning (ML) models are used to automate data processing and mesh generation associated with the models and methods disclosed herein. In some embodiments, a variety of manual and semi-automated tools can be used to process the data and generate meshes. In some embodiments, ML is used to streamline data processing and mesh generation to minimize the manual labor required. In some embodiments, the ML models process dicom files obtained from CT imaging. In some embodiments, if MRI data is obtained, the MRI image is used by one or more ML models for generating a mesh of the myocardium.

In another aspect, ML is used to significantly improve the speed/efficiency of the computational biophysics model. In some embodiments, the ML model is trained based on the inputs and outputs of the biophysics model with the goal of using the ML model in lieu of the biophysics model. In one aspect, the advantage of an ML model is primarily speed, as the algebraic ML model can be easily parallelized and run on GPUs. In some embodiments, the ML model does not require the numerical integration of a system of PDEs which potentially create a computational bottleneck.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are examples only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separately from other components and processes described herein. Each component and process can also be used in combination with other assembly packages and processes. The present aspects may enhance the functionality and functioning of computers and/or computer systems.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

A workflow was developed (see FIG. 5) for semi-automated 3D mesh generation from cCTA required for CFD modeling of epicardial flow. The workflow comprises 3 main steps which include: 1) coronary artery segmentation; 2) surface mesh generation; and 3) 3D volumetric mesh generation. In all 3 steps, open-source software along with Python scripts for automation wherever possible are implemented. In step 1, coronary segmentation is done using the voxel-based tools available in 3D Slicer. In brief, we applied a variety of thresholding, filtering, and smoothing functions to segment the epicardial artery lumens. Despite the availability of automated tools such as growing from seeds, these algorithms did not perform well on small coronary artery branches, and it was observed that semi-automated threshold-based methods which required some manual tracing of vessels worked best. The segmented data was post-processed and surface meshes were generated for step 2. The surface meshes were further processed in VMTK, an open-source vascular modeling library. VMTK was used to automatically generate centerlines and to create boundary and surface patches which we used for assigning boundary conditions for the CFD models and in the mesh refinement algorithm in step 3. Specifically, each surface patch is associated with a minimum distance to the centerline which is used to assign a maximum cell size to each individual vessel segment during volume mesh generation. Finally, a 3D volume mesh was generated in step 3 using the free software cfMesh. Most of the parameters required by cfMesh were obtained automatically from the processing done in step 2, so this final step was fully automated. An example mesh from one of the cCTA datasets for which PET and catheterization data was also obtained is shown in FIG. 5.

For this pilot project, 8 (with plans to collect a total of 35) hybrid PET/cCTA examinations performed simultaneously on patients on the same cardiac-capable PET/CT scanner for research were analyzed. In each of these research patients, all also had coronary catheterization, and those with epicardial lesions identified also had either FFR or iFR performed and intracoronary pressure waveform measurements recorded. In addition, as part of this pilot project, data from ˜5000 cCTA and 350 PET/CT datasets obtained from clinical examinations were used. Of these, ˜500 have had coronary catheter angiography performed within a 1-2 year timespan, and ˜30 patients have had both cCTA and PET examinations, 15 of which also had catheterization within a 1-2 year timespan. In all patients with catheterization, we obtained pressure waveform measurements along with measurements of either FFR or iFR for stenoses.

### Example 2

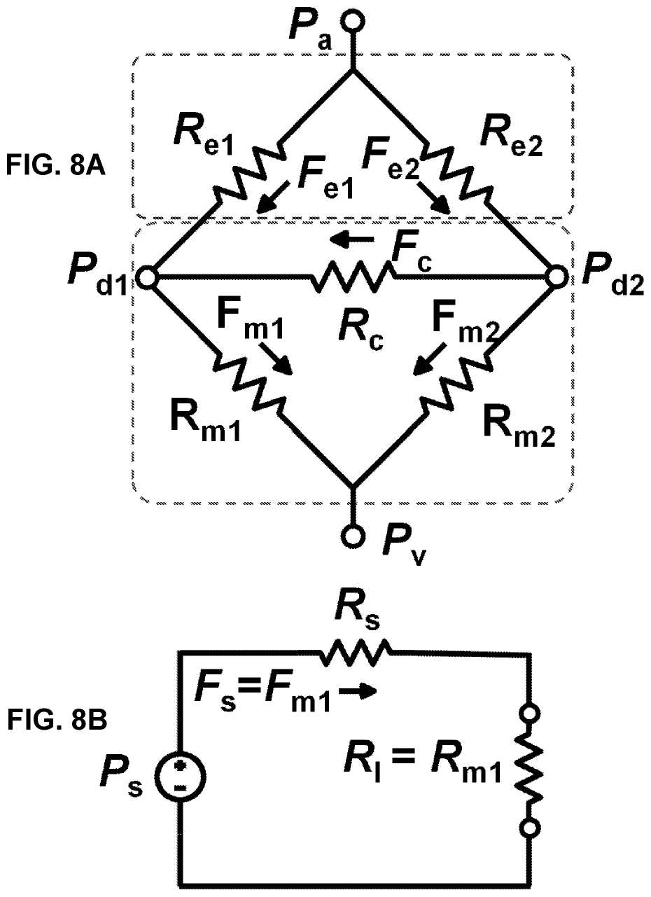

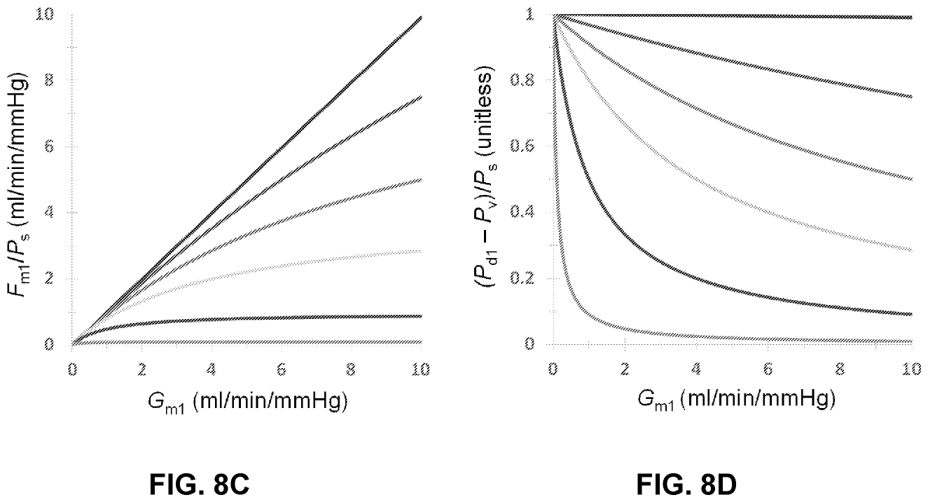

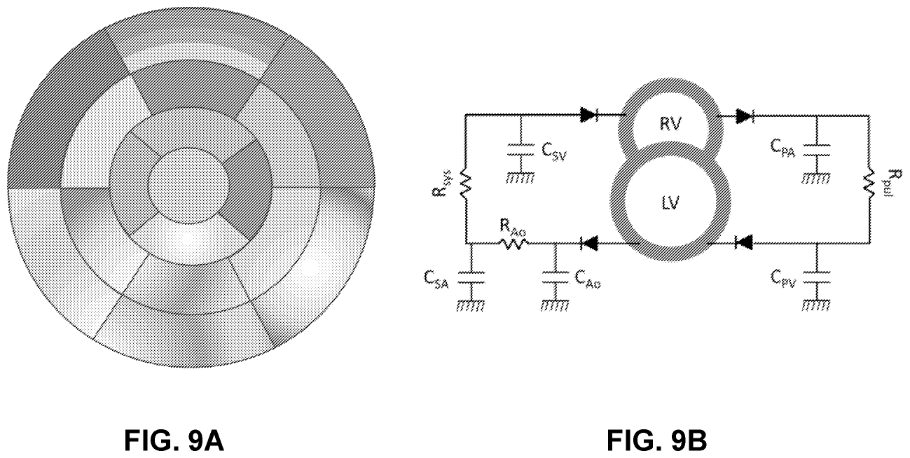

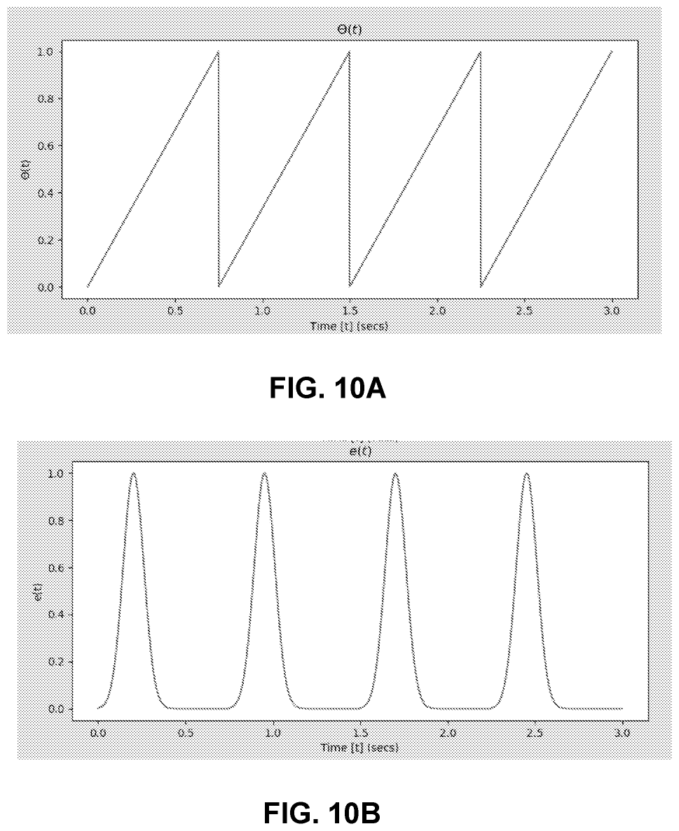

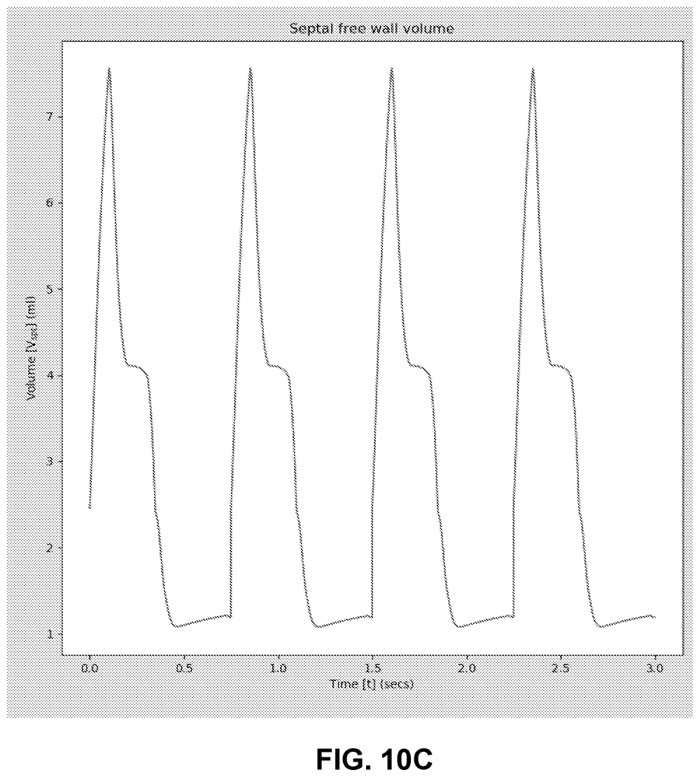

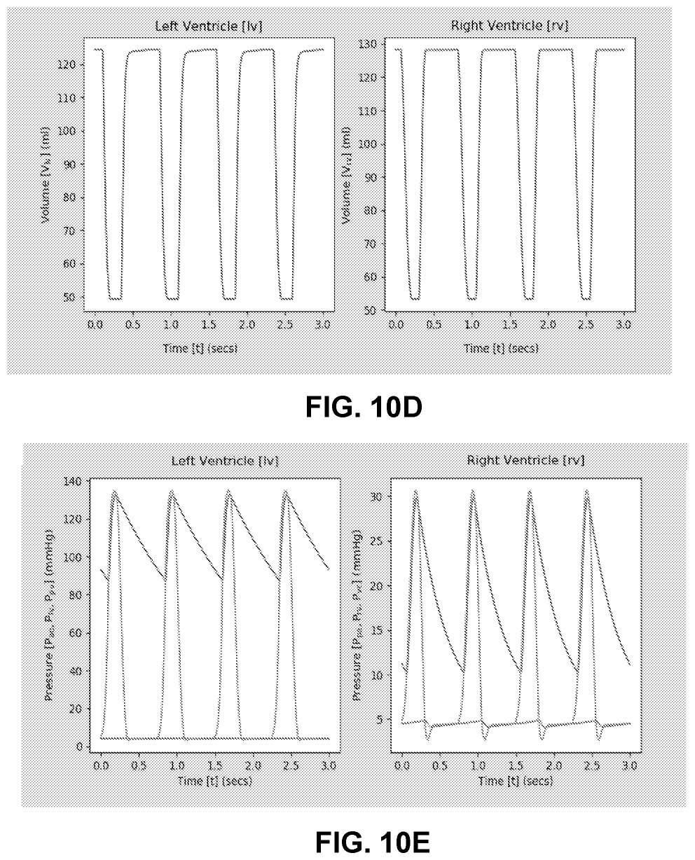

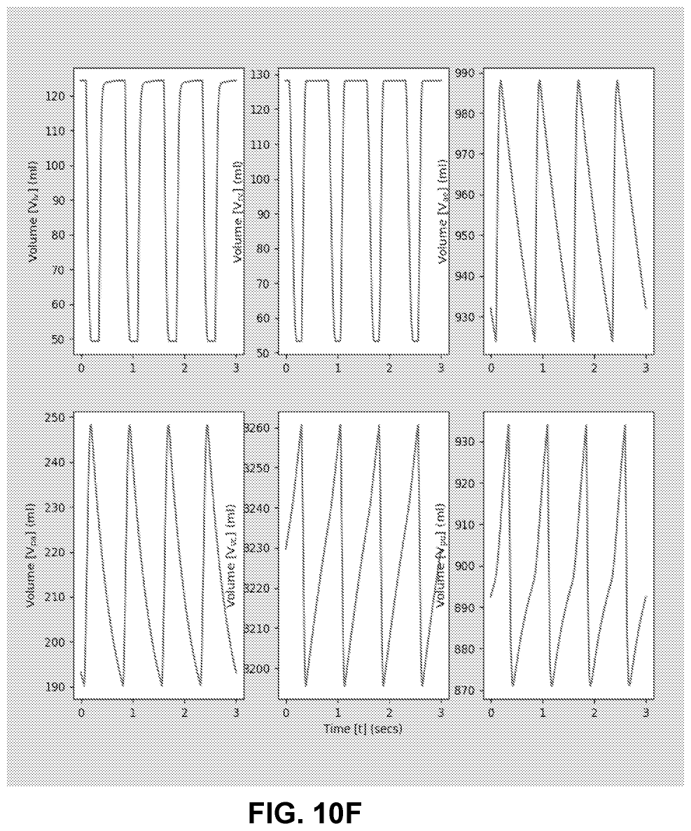

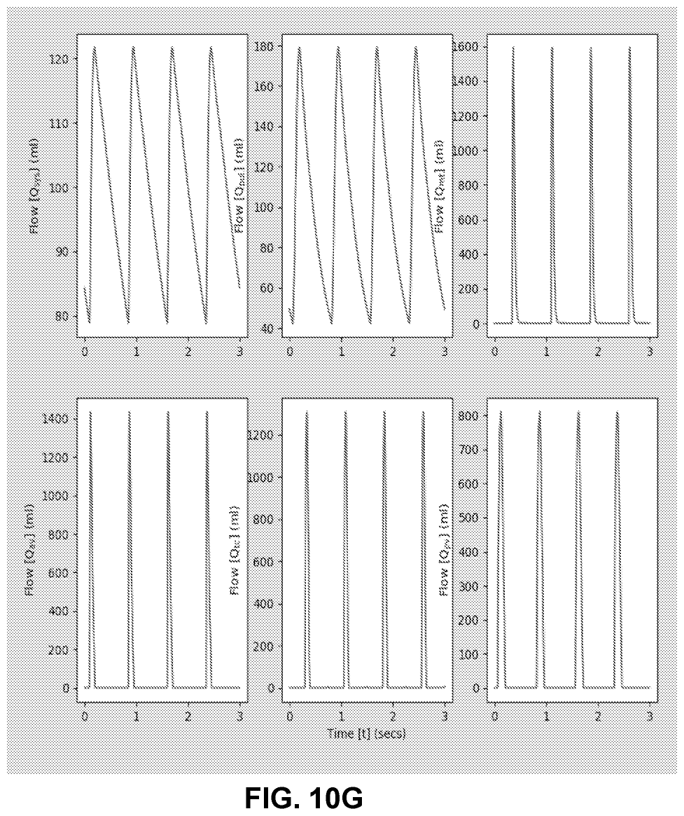

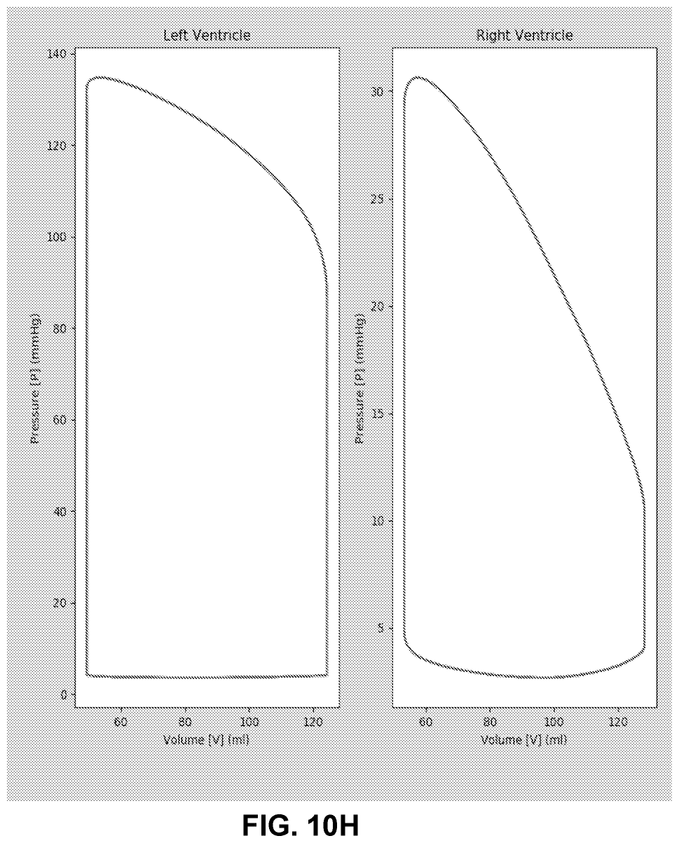

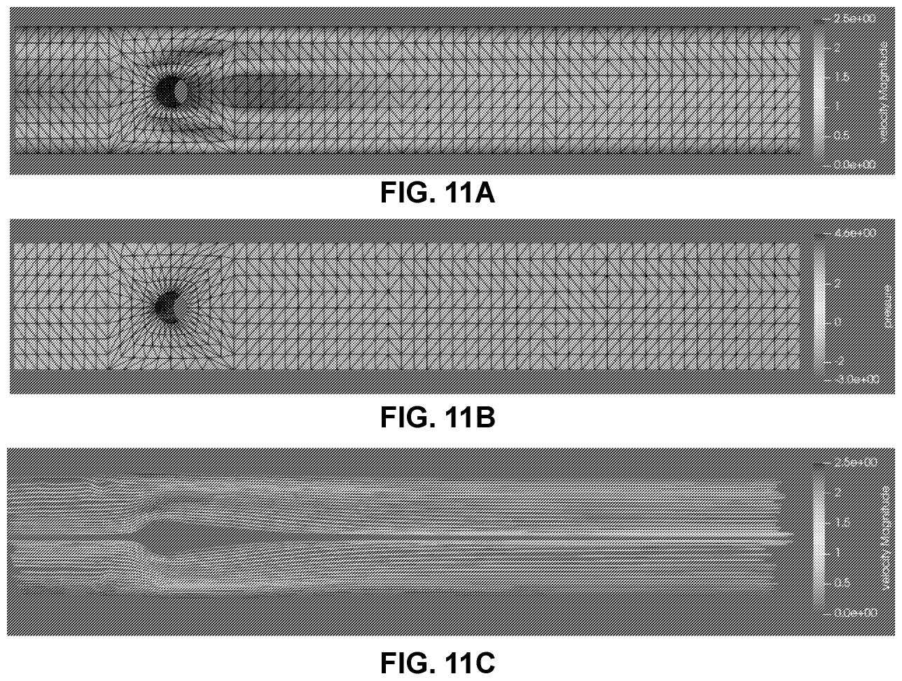

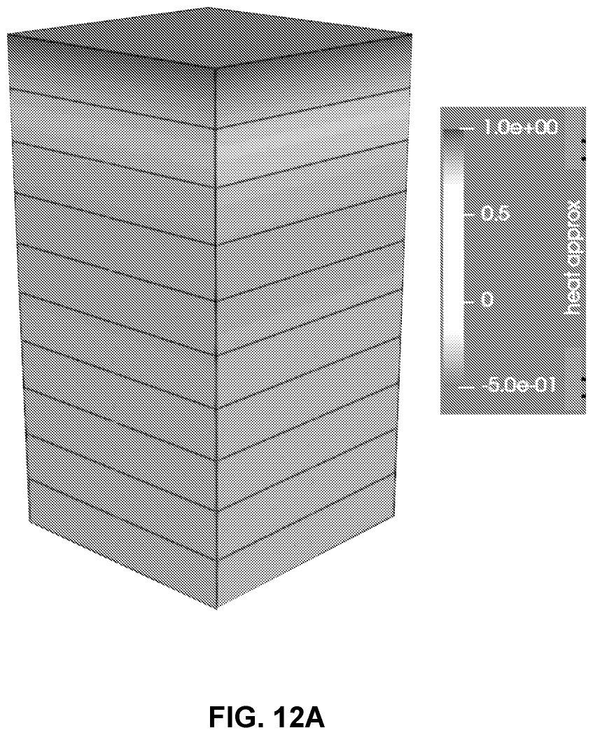

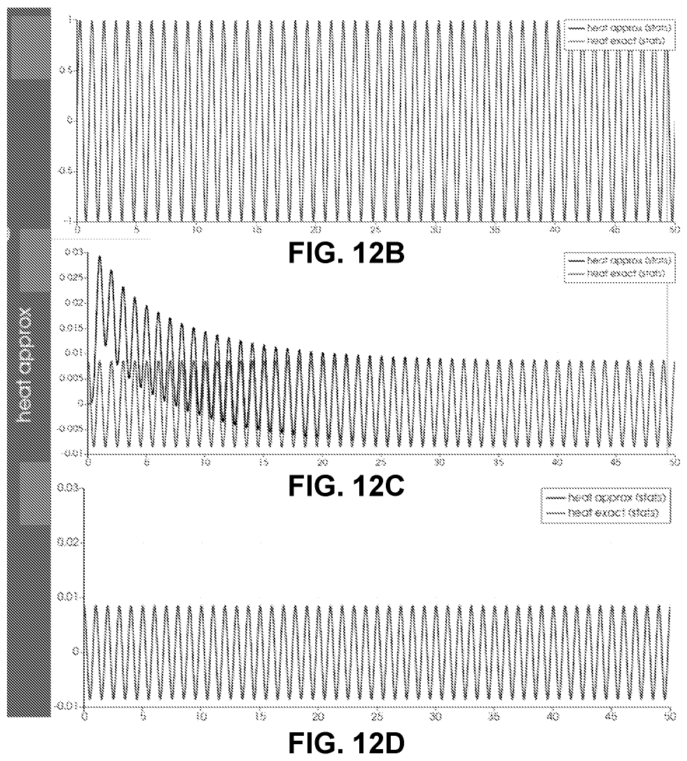

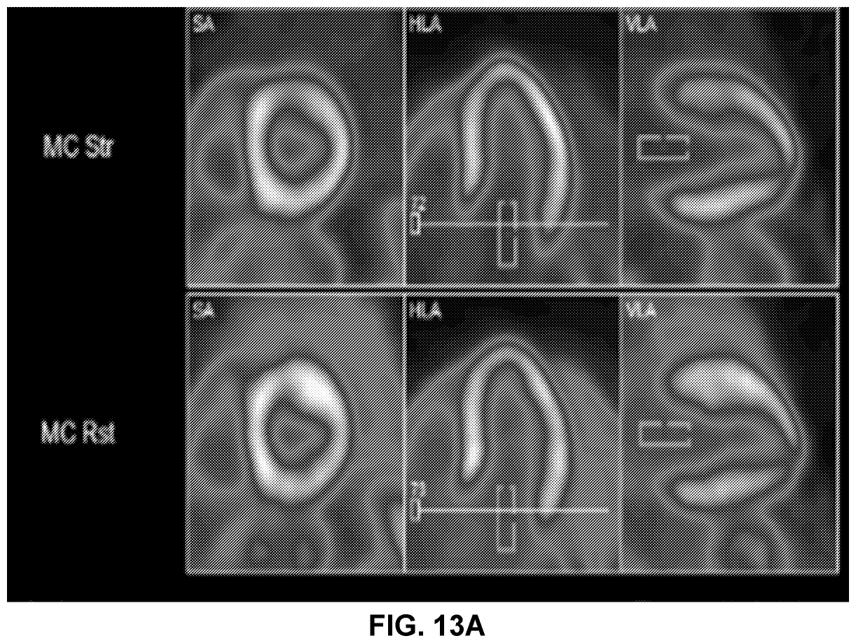

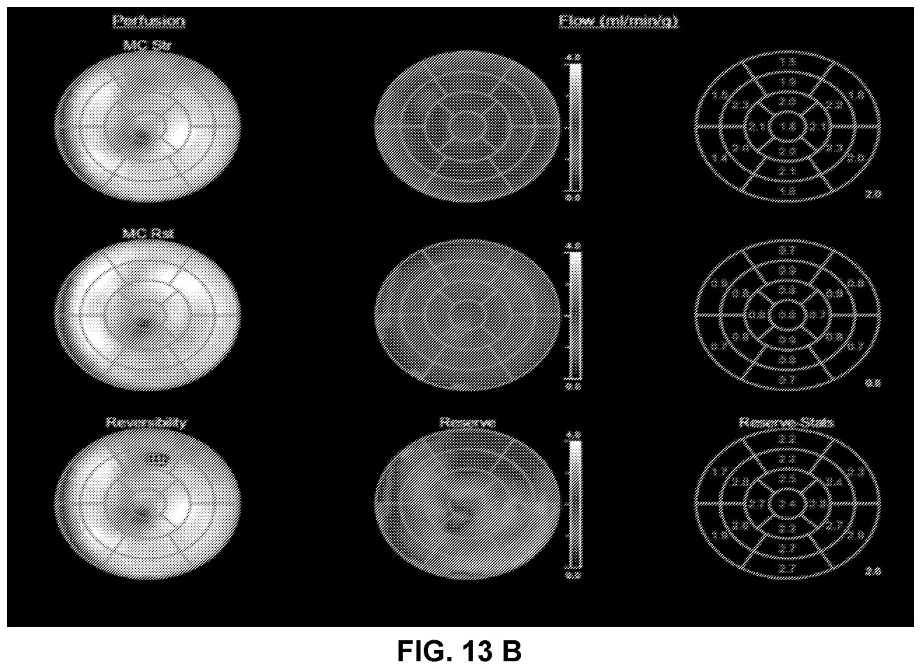

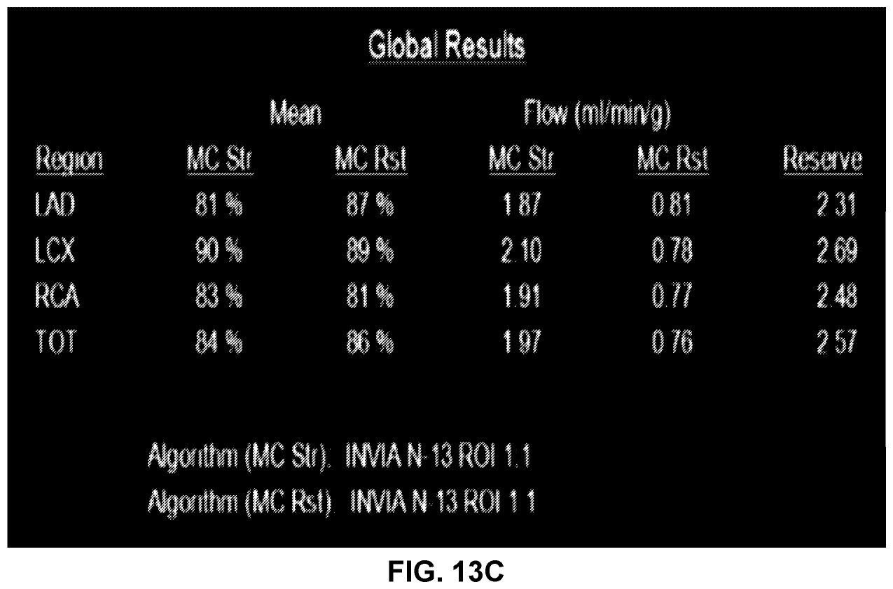

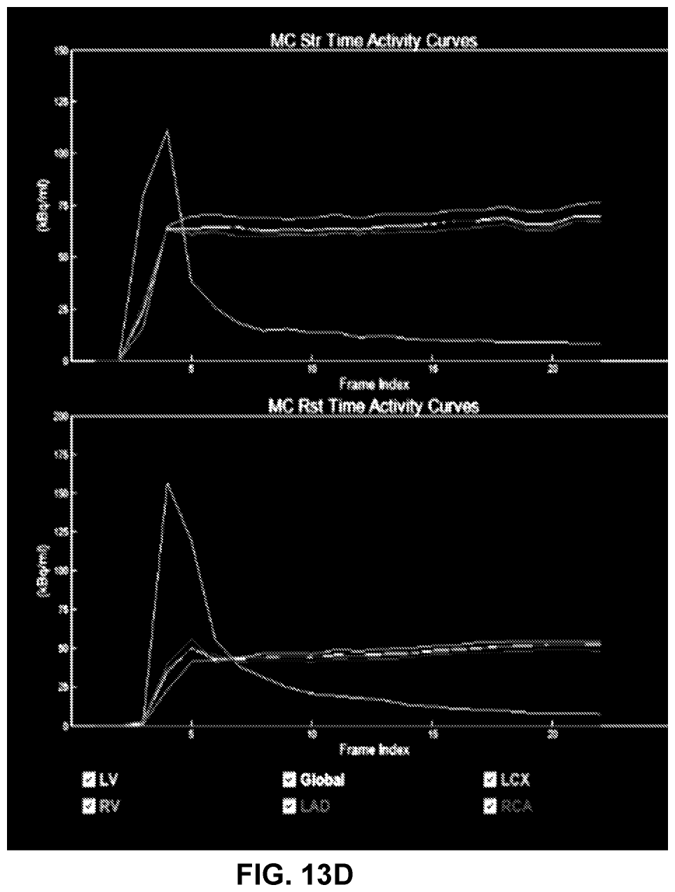

To better illustrate the concepts underlying APR and its measurement, a simple 2-vessel resistive network model of coronary circulation (a generalized form of that proposed by Pijls et al.), shown illustrated in FIG. 8A, is considered in deriving equations that are commonly used to estimate FFR from pressure wire measurements. In this example, the influence of the entire network on myocardial flow Fₘ₁ through microvascular bed m1 is characterized. Some insight can be gained by expressing circuit A in terms of an equivalent source-load circuit using basic techniques in linear circuit analysis as shown in FIG. 8B. Fₘ₁ can then readily be derived from expressions for equivalent source pressure Pₛ and equivalent source resistance Rₛ via Ohm's law as Fₘ₁=Pₛ/(Rₛ+Rₘ₁).

Generally, microvascular arteriolar resistance varies dynamically and is controlled by a variety of physiological mechanisms to match myocardial perfusion with myocardial work rate, the success of which depends critically on the sensitivity of changes in flow to changes in resistance. By plotting microvascular flow Fₘ₁ against microvascular load conductance Gₘ₁=Rₘ₁−1 as shown in FIG. 8C, an important property emerges in which the sensitivity of Fₘ₁ to changes in Gₘ₁ becomes increasingly blunted as the ratio of Gₘ₁:Gₛ increases. The corollary is that the ability of the coronary system to effectively regulate perfusion is lost at increased workloads in the presence of sufficiently severe upstream stenosis, a phenomenon that is well documented clinically. We can also plot (P_{d1}−Pᵥ)/Pₛ (which represents the equation derived by Pijls et al. to estimate FFR when Pₛ=Pₐ−Pᵥ) against Gₘ₁ as shown in FIG. 8D to see how FFR is affected by the Gₘ₁:Gₛ ratio. Note that the derivation of FFR in the analysis by Pijls et al. relies on the assumption that Rₑ₁=0 in the absence of a stenosis; if this does not hold, FFR becomes a much more complicated expression involving all resistances in the network and cannot be determined by a single measurement independent of the network. Likewise, their derivation for collateral flow F_{c} depends on the additional assumption that Rₑ₂=0 which cannot be applied in the general case of multivessel disease. The network behavior of the coronary system and how network properties and microvascular remodeling contribute to ischemic heart disease remains uncharacterized.

About a century since Thoma first demonstrated the adaptation of vessels to flow and Murray first postulated a central role of shear stress, distinct modes of microvascular remodeling including angiogenesis, arteriogenesis, and pruning are now recognized. Angiogenesis and pruning refer to the sprouting and rarefaction of existing capillary beds, respectively, whereas arteriogenesis refers to the luminal growth of existing genetically determined native collateral arteries connecting microvascular beds. The functional importance of these processes in ischemic heart disease remains poorly understood. While learning whether and how this sort of network-level remodeling occurs is an interesting and important scientific question in and of itself, accurate characterization of remodeling is essential in any method seeking to quantify APR, as myocardial perfusion Q is a function of not only volumetric flow Fₘ₁ through a microvascular bed, but also of the distribution of Fₘ₁ and adjacent capillary bed Fₘ₂ within the myocardium, which depends itself on microvascular properties such as capillary density and spatial distribution and arteriolar resistance. This relationship can be expressed mathematically for the 2-vessel coronary system in FIG. 8A as

$$\overline{Q} ( v ) = ( {F}_{e 1} + {F}_{c} ) {\overline{ρ}}_{{G}_{m 1}} ( v ) / {G}_{m 1} + ( {F}_{e 2} - {F}_{c} ) {\overline{ρ}}_{{G}_{m 2}} ( v ) / {G}_{m 2} \qquad ( 1 )$$

where v represents a particular voxel, Q(v) represents average perfusion within that voxel, ρ̅_{Gm,x}(v) represents the average conductance density of microvascular bed x within voxel v (where ρ_{Gm,x}(p)∝capillary density of microvascular bed x at point p), and the F's and G's correspond to the network level flows and conductances defined in FIG. 8. If both the network level parameters and microcirculatory parameters are known, then Eq. 1 can be solved to obtain APR at any voxel v or over any segment S of the myocardium (corresponding to a set of voxels) associated with an epicardial lesion at e1 according to

$$APR = ( {\sum }_{v ∈ S} \overline{Q} ( v ) m ( v ) \vert {R}_{e 1 , N} - {\sum }_{v ∈ S} \overline{Q} ( v ) m ( v ) \vert {R}_{e 1} ) / {\sum }_{v ∈ S} m ( v ) \qquad ( 2 )$$

where m(v) is the myocardial mass of voxel v, and Rₑ₁ and R_{e1,N} correspond to resistance in epicardial artery e1 with stenosis present and following treatment of the stenosis, respectively, during maximum hyperemia.

To illustrate the fundamental limitations of FFR and differences between APR and FFR, consider the scenario in which the FFR of vessel e1 is estimated by pressure wire to be 0.5, corresponding to a Gₘ₁:Gₛ ratio of 1:1. This could correspond to any of the curves in FIG. 8D, and without knowing which curve it is, absolute flow reserve cannot be estimated. For example, assume the stenosis is treated by PCI improving FFR to ˜1.0, corresponding to the top curves in FIG. 8. If the vessel were initially on the middle curves, then as shown by FIG. 8C, Fₘ₁ would increase from 5 to 10 corresponding to an absolute flow reserve of 5, whereas if it were on the curves second from the bottom, then Fₘ₁ would increase from 0.5 to 1 corresponding to an absolute flow reserve of only 0.5. However, even if absolute flow reserve were known, Eqs. 1 and 2 show that one also needs to know the distribution of the reserve flow within the myocardium to determine APR, and this distribution depends on properties of the entire coronary network as well as patient-specific epicardial and microcirculatory coronary anatomy. The curves also illustrate how FFR can potentially be misleading in the setting of microvascular disease, which would manifest as lower values of Gₘ₁. In general, the values of FFR may be higher for a given epicardial stenosis in the setting of microvascular dysfunction, and the degree of perfusion impairment due to a stenosis may be greater than what FFR might indicate. Thus, APR could be particularly important in evaluating the functional severity of a lesion in the setting of microvascular disease.

### Model Development

The above analysis demonstrates how the properties of both macrocirculation and microcirculation contribute to perfusion and APR. However, the relationship described in Eq. 1 assumes the extremely simplified and unrealistic resistive network model of the coronary system shown in FIG. 8 that does not account for the pulsatile nature of coronary perfusion, ventricular-vascular interactions, the effect of those interactions on transmural gradients on perfusion, or individual patient anatomy and topology of the coronary epicardial arteries and microcirculation. The goal of this Example is to develop a framework for extracting patient-specific physiologically based and anatomically constrained models of coronary network dynamics and myocardial perfusion accounting for all these effects directly from quantitative imaging data.

The mathematics and associated numerical methods of coronary network models are well established and generally invoke formulations of the epicardial conduit arteries based on either OD lumped parameter RLC circuit representations or 1 D or 3D representations based on the Navier-Stokes equations. Parameters of these models can be extracted directly from the anatomic information provided in coronary CTA as done with FFRCTA. The boundary condition at the coronary inlet can be estimated directly e.g. using MRI measurement of the aortic flow waveform and/or measuring brachial artery pressure. However, the outflow boundary conditions cannot be easily measured non-invasively and have typically relied on either data in the literature or on a variety of heuristic assumptions (such as coronary flow proportional to myocardial mass and microvascular resistance proportional to epicardial branch diameter); none have accounted for patient-specific properties of the microcirculation. The fundamental strengths of the approach are twofold: 1) the ability to leverage quantitative perfusion data to develop patient-specific models of the microcirculation that can directly be used to derive the outflow boundary conditions for each terminal epicardial branch vessel; and 2) the ability to account for the spatial distribution of flow, ventricular-vascular interactions contributing to transmural effects on flow and resulting differential vulnerability of the subepicardium and subendocardium to ischemia, and microvascular remodeling, which are all necessary to estimate APR.

Patient-specific models of the aortic root and epicardial coronary arteries can be developed based on the 3D Navier-Stokes equations using the open-source image-based computational hemodynamics software CRIMSON, with inflow and outflow boundary conditions to the aortic root provided by a varying elastance model of the heart and lumped compartment model of the peripheral circulation, respectively, and outflow boundary conditions of each terminal epicardial artery branch provided by a lumped parameter model of the coronary microcirculation accounting for intramyocardial pressure effects on coronary flow. The microcirculatory model will also account for the possibility of pre-arteriolar collateral flow between epicardial outlet nodes in the form of lumped parameter connections between the nodes. The spatial distribution of flow, or flow density, of each microvascular bed within the myocardium will represent a novel feature, and in general, will be accounted for using spatial distributions of impedance with the relationship between the lumped parameter microcirculatory model and the impedance distribution functions provided by theory from electrical circuit networks. The idea is similar to that used in electrical impedance tomography (EIT) whereby unknown spatial distributions of impedance within the body are estimated, except here the input represents outlet pressure at each terminal epicardial branch rather than current injected through skin electrodes, the data represents perfusion at each voxel in the myocardium rather than surface potential at each skin electrode, and the governing model represents the lumped parameter microcirculatory model rather than Maxwell's equations. As in EIT, estimating the spatial distribution of microvascular impedance within the myocardium is an ill-posed problem and represents a major challenge. However, this can be overcome by applying various methods for regularization, for example by incorporating prior information to reduce the dimension of the parameter space (e.g. by accounting for known microvascular branching patterns), along with other methods including techniques for spatial regularization and minimization in total variation in parameters.

Even if the problem becomes well-posed through regularization, it can still be ill-conditioned in that solutions may be sensitive to errors in the data. Any imaging modality is an imperfect representation of reality. For example, PET is affected by various artifacts including positron range, scatter, attenuation, and detector blur that introduce noise and blurring in the images. The resolution limits of PET may limit the ability of the model to discriminate fine details in the parameters, e.g. transmural gradients in parameters predisposing to subendocardial ischemia. These issues can be investigated by performing a sensitivity analysis of model solutions to perturbations in the data. If it is determined that robust solutions cannot be determined using PET data alone, these studies could reveal what additional clinical/imaging data may be needed to achieve a robust solution. However, stable solutions do not necessarily mean accurate solutions. Any systematic errors in the model can result in predictions that differ from clinical observations. The validity of the model can be tested in several ways. One would be to compare APR estimated from the model with that calculated directly using data from PET obtained prior to and immediately following invasive therapy. Comparisons can also be made of predicted measurable variables from unrelated modalities performed on the same patient, e.g. epicardial pressure and flow waveforms measured during coronary catheterization. In addition to PET, MR myocardial perfusion can be performed including PET/MR perfusion. In some embodiments, the added spatial resolution provided by MR perfusion can lead to improved identifiability of spatially dependent parameters in the model.

### Example 3

A good physiological index for myocardial ischemia is predictive of treatment response, can be measured non-invasively, and correlates as closely to myocardial ischemia as possible. Myocardial O₂ concentration is the best parameter but is often difficult to measure. The second best parameter is the second best parameter, as it correlates directly with O₂ delivery. A good physiological index also uses a minimal number of assumptions and replicates underlying physiology as closely as possible, has an accuracy that does not depend on the absence of other comorbidities and is equally accurate in patients with microvascular disease as in patients with normal microcirculation.

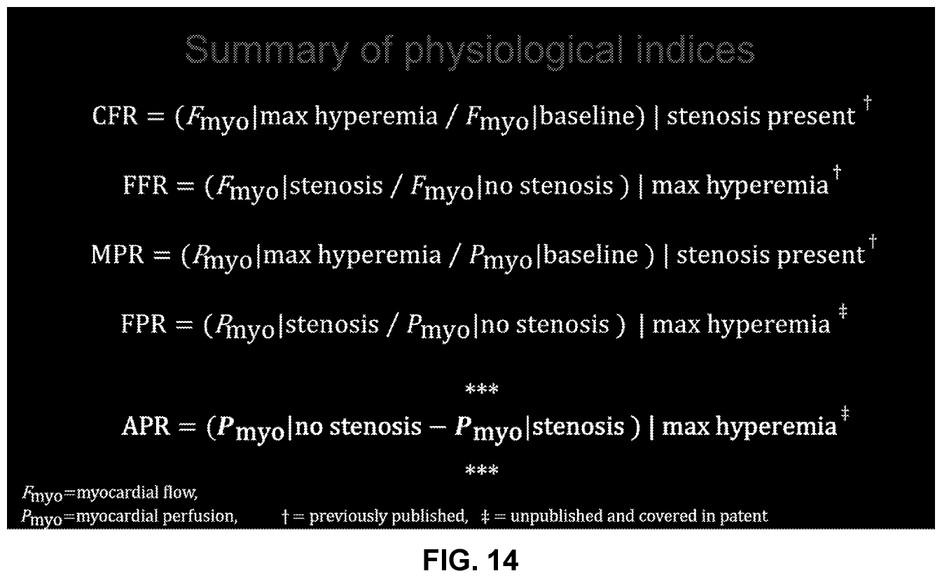

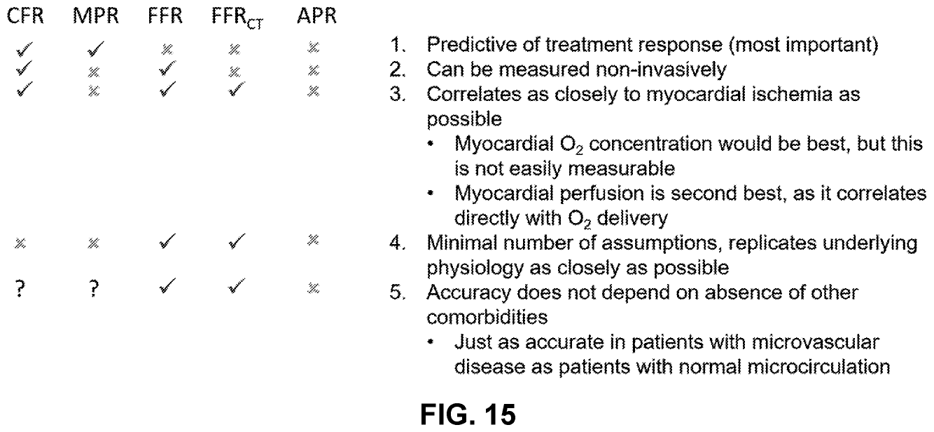

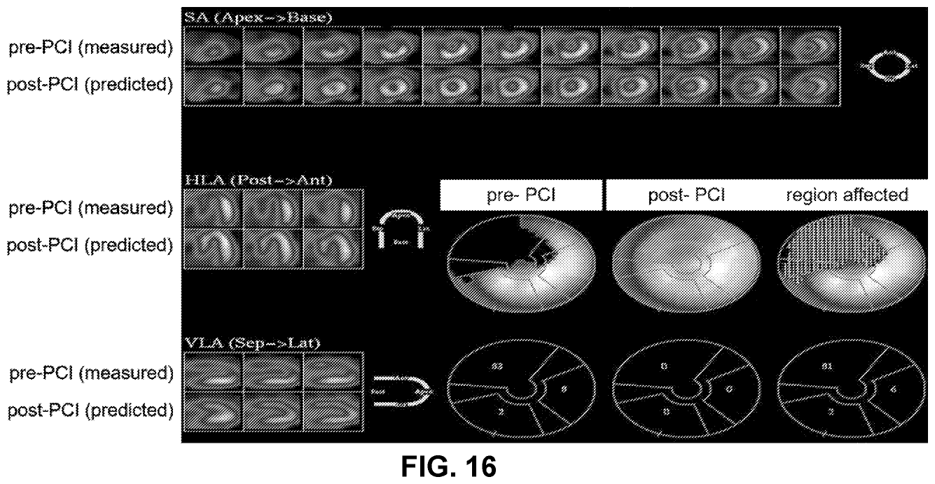

FFR-CT is not an ideal physiological index since it is a crude model, requiring additional non-physiological assumptions including baseline total coronary flow is proportional to LV mass according to F˜M^{0.75}, microvascular resistance downstream of an epicardial vessel is proportional to its lumen cross-sectional area, and microcirculatory resistance decreases predictably in maximum hyperemia. A summary of various related indices can be seen in FIGS. 14 and 15.

Absolute Perfusion Reserve (APR) is a physiological index rooted in the fundamental physiological principle that the severity of a stenosis is directly related to the degree of perfusion deficit attributable to the stenosis. In particular, lesions with large perfusion deficit would likely benefit from PCI, whereas lesions with small perfusion deficit would likely not, and therefore APR provides a means to predict perfusion improvement with treatment. APR is a fully non-invasive measurement that combines the best features of myocardial perfusion imaging (e.g. with PET, MRI, CTA) with anatomic imaging of coronary CTA in a synergistic way. APR has far fewer assumptions than FFR and FFR_{CT}. While it still requires a model to predict perfusion in the absence of stenosis, much like FFR requires a model to predict flow in the absence of stenosis, the combination of anatomic and perfusion data of APR allows for a more realistic model that avoids the need for many of the assumptions required in FFR and FFR_{CT}. The technology also provides a means to detect and quantify microvascular disease non-invasively, which can play an important role in identifying and directing medical therapy for patients without significant large vessel stenosis who have microvascular disease.

APR is calculated as the difference between the predicted post-PCU perfusion map and the measured pre-PCI perfusion map. It can be defined globally, regionally (e.g. for vessel-specific regions), and on a voxel-wise basis, which is represented in FIG. 16.

APR can be used for guiding PCI placement. The method begins with a patient obtaining myocardial perfusion imaging along with coronary CTA (cCTA), for example, a combined PET/cCTA or combined CT perfusion/cCTA. Then imaging data is processed using the machine learning algorithms described in the present disclosure, and APR is computed using the physiologically realistic biophysics model. The results are provided to a clinician in an easy-to-use software providing tools for analysis. A 3D model of epicardial arteries and myocardium can be visualized, rotated, and manipulated in 3D space. An epicardial stenosis or stenosis is identified and selected for virtual PCI. Selection for stenosis can be chosen manually or automated based on calculated criteria such as percent luminal stenosis or predicted FFR, which are easily visualized using color coding of the epicardial artery model. APR is then calculated based on the selected stenosis or stenosis. Calculated values include global APR (based on the entire myocardium), vessel-specific APR (based on segments of myocardium supplied by individual vessels), and voxel-wise APR. Voxel-wise APR is visualized using color coding superimposed on the 3D myocardium model, and voxel-wise APR statistics such as minimum, maximum, mean, or other related parameters can be computed over manually selected regions of the myocardium to help identify regions of ischemic vulnerability. Finally, the decision for PCI is made by a clinician based on the APR analysis.

APR can also be used to help guide medical management. A powerful feature of the sophisticated microvascular modeling required of APR is that it can also be used to detect and quantify microvascular disease. Microvascular properties such as vessel density, collateral density, and impedance can be quantified on a voxel-wise basis. In much the same way APR is calculated from predicted response to PCI, APR can also be calculated from predicted response to a variety of medical therapies. This can include, but is not limited to, perfusion response to normalization of microvascular function by therapies such as ACE inhibitors, perfusion response to normalization of LV end diastolic pressure (e.g. through improved diastolic function with beta-blockers, or by reducing afterload with blood pressure medications), and perfusion response to reduced venous pressure through diuresis and inotropic agents. Since the model includes ventricular mechanics and whole-body hemodynamics, a variety of in-silico therapies like these can be performed and used to assess perfusion response. The clinician can use this information in decisions for PCI in patients with comorbidities, for example, if APR related to stenosis is large and APR related to microvascular function is small, this would indicate a higher relative benefit of PCI versus medical therapy. On the other hand, if APR related to stenosis is small and APR related to microvascular function is large, less benefit of PCI would be indicated and medical management alone would be favored. Additionally, follow-up scans can be used to assess treatment response over time.

APR provides a nearly ideal physiological metric for guiding coronary interventions and medical therapy, with many theoretical and practical advantages over other metrics including the gold standard FFR. It is predicted that APR can outperform and ultimately replace all currently available technology in predicting treatment response and selecting patients for intervention, as well as disrupt current workflow for assessing ischemic heart disease. The current standard workflow includes cCTA, followed by FFR_{CT}, followed by invasive coronary angiography and invasive FFR, and finally real-time decision for PCU while the patient is on the cath table. The workflow of the present disclosure includes combined cCTA and myocardial perfusion imaging, APR analysis, and a decision for PCI versus medical management made without the need for further evaluation with a cath. The clinician can carefully assess all options and plan an optimal therapy prior to ever taking the patient to the cath lab.

Overall APR is a technology grounded in fundamental physiological principles and state-of-the-art mathematical modeling. APR provides a nearly ideal physiological metric for guiding coronary interventions and medical therapy. It can predict treatment response to a variety of invasive and medical therapies, can be measured non-invasively, is based on measurement of perfusion which is currently the best available non-invasive biomarker of ischemia, has a minimal number of assumptions required (far less than the gold standard FFR and its non-invasive surrogate FFR_{CT}, and its results are not biased by the presence of comorbidities such as microvascular disease. APR is the only available technology that can non-invasively detect and quantify microvascular disease and offers the potential to supplant FFR and disrupt existing workflows for the evaluation and management of patients with coronary ischemia.

What is claimed is:

1. A computer-implemented method for selecting a treatment for a subject with an epicardial stenosis, the method comprising:
a. providing, to a computing device, a multi-level perfusion model configured to estimate a myocardial perfusion for the subject, wherein the multi-level perfusion model comprises a 3D epicardial mesh representative of an aortic root, left and right coronary arteries, and associated epicardial branches of the subject, wherein the 3D epicardial mesh further comprises the epicardial stenosis;
b. estimating, using the computing device, an occluded myocardial perfusion using the multi-level perfusion model;
c. modifying, using the computing device, the 3D epicardial mesh to represent the treatment of the stenosis;
d. estimating, using the computing device, a post-treatment myocardial perfusion using the modified multi-level cardiac perfusion model;
e. estimating, using the computing device, an absolute perfusion reserve (APR) based on the estimated occluded myocardial perfusion and post-treatment myocardial perfusion; and
f. selecting, using the computing device, a treatment for the subject if the APR is greater than a threshold value.

2. The method of claim 1, wherein absolute perfusion reserve (APR) is estimated according to the equation:
$$APR = ( {\sum }_{v ∈ R} \overline{Q} ( v ) m ( v ) \vert {Ω}_{e 1 , N} - {\sum }_{v ∈ R} \overline{Q} ( v ) m ( v ) \vert {Ω}_{e 1 , S} ) / {\sum }_{v ∈ R} m ( v )$$
wherein Σ_{v∈R}Q̅(v)m(v)|Ω_{e1,N} and Σ_{v∈R}Q̅(v)m(v)|Ω_{e1,S} represent the estimated post-treatment and occluded myocardial perfusions, respectively, v∈R represents a voxel v within a region R of a myocardium, Q̅(v) represents an average perfusion within the voxel v, m (v) represents a myocardial mass of voxel v, and Ω_{e1,S} and Ω_{e1,N} correspond to the 3D meshes with the stenosis and with the stent, respectively, during maximum hyperemia.

3. The method of claim 1, wherein the multi-level perfusion model further comprises a coronary microcirculation model configured to estimate the myocardial perfusion, wherein the coronary microcirculation model is operatively coupled to the 3D epicardial mesh at a plurality of epicardial outflow nodes defining a myocardial perfusion region, the coronary microcirculation model comprising:
a. a spatially-lumped model representative of average or layer-wise perfusion over each myocardial perfusion region; or
b. a finite element mesh representative of voxel-wise perfusion over each myocardial perfusion region.

4. The method of claim 1, wherein the multi-level perfusion model further comprises a varying-elastance heart model operatively coupled to the aortic root representation of the 3D epicardial mesh, the varying-elastance heart model configured to provide an elastance driving function to drive a representation of pulsatile blood pressure and flow within the multi-level perfusion model.

5. The method of claim 1, wherein the multi-level perfusion model further comprises a lumped-compartment model of peripheral circulation operatively coupled to the aortic root representation of the 3D epicardial mesh, the lumped-compartment model of peripheral circulation configured to represent systemic and pulmonary resistances, compliances, and inertances used to drive the representation of pulsatile blood pressure and flow within the multi-level perfusion model.

6. The method of claim 1, wherein the multi-level perfusion model further comprises a Voronoi model configured to define the myocardial perfusion regions.

7. The method of claim 1, further comprising producing, using the computing device, the multi-level perfusion model based on medical imaging data, the medical imaging data comprising cCTA data, MRI data, PET perfusion data, and any combination thereof.

8. The method of claim 7, wherein producing the multi-level perfusion model further comprises:
a. receiving, at the computing device, cCTA data;
b. segmenting, using the computing device, the cCTA data to produce a 3D representation of the epicardial blood vessels; and
c. transforming, using the computing device, the 3D representation of the epicardial blood vessels into the 3D epicardial mesh.

9. The method of claim 7, wherein producing the multi-level perfusion model further comprises:
a. receiving, at the computing device, cCTA data; and
b. transforming, using the computing device, the cCTA data into a 3D epicardial mesh using a machine learning model.

10. The method of claim 9, wherein the machine learning model comprises a convolutional neural network.

11. The method of claim 10, wherein the convolutional neural network is a graph convolutional network.

12. The method of claim 7, wherein producing the multi-level perfusion model further comprises:
a. receiving, at the computing device, the PET perfusion data;
b. defining, using the computing device, a plurality of perfusion parameters defining the coronary microcirculation model to match the PET perfusion data.

13. The method of claim 12, wherein defining the plurality of perfusion parameters to match the PET perfusion data further comprises:
a. assigning, using the computing device, an initial set of perfusion parameters to an AHA 17-segment model; and
b. refining, using the computing device, the initial set of perfusion parameters of the AHA 17-segment model using Voronoi partitioning with weighted Voronoi diagrams to account for patient-specific epicardial coronary anatomy.

14. The method of claim 12, wherein defining the plurality of perfusion parameters defining the coronary microcirculation model to match the PET perfusion data further comprises:
a. receiving, using the computing device, cCTA data and PET perfusion data; and
b. transforming, using the computing device, the cCTA data and PET perfusion data into a detailed model of the microcirculatory network using space-filling fractals and constrained constructive optimization.

15. The method of claim 1, wherein modifying the 3D epicardial mesh to represent the treatment of the stenosis comprises replacing, using the computing device, a region of the 3D epicardial mesh representative of the stenosis with a substitute region representative of:
a. the region with blockage associated with the stenosis removed; or
b. the region modified to represent an implanted stent.

16. The method of claim 1, further comprising:
a. modifying, using the computing device, the 3D epicardial mesh to represent an implantation of a first stent as a first treatment of the stenosis;
b. estimating, using the computing device, a first post-treatment myocardial perfusion using the modified multi-level cardiac perfusion model;
c. modifying, using the computing device, the 3D epicardial mesh to represent an implantation of a second stent as a second treatment of the stenosis;
d. estimating, using the computing device, a second post-treatment myocardial perfusion using the modified multi-level cardiac perfusion model;
e. estimating, using the computing device, a first absolute perfusion reserve (APR) based on the estimated occluded myocardial perfusion and first post-treatment myocardial perfusion;
f. estimating, using the computing device, a second absolute perfusion reserve (APR) based on the estimated occluded myocardial perfusion and second post-treatment myocardial perfusion; and
g. selecting, using the computing device, a treatment for the subject associated with the higher of the first and second absolute perfusion reserves (APRs).
